(19) United States
(12) Patent Application Publication
(10) Pub. No.: US 2018/0171411 A1
(43) Pub. Date: Jun. 21, 2018

(54) GFRalpha1 AS A BIOMARKER FOR CISPLATIN-INDUCED CHEMORESISTANCE AND METASTASIS

(71) Applicants: Dae Joon KIM, San Antonio, TX (US); Mihwa KIM, San Antonio, TX (US); Thomas J. SLAGA, San Antonio, TX (US); Won Jae KIM, Gwangju (KR); Jin-Yeon JUNG, San Antonio, TX (US)

(72) Inventors: Dae Joon KIM, San Antonio, TX (US); Mihwa KIM, San Antonio, TX (US); Thomas J. SLAGA, San Antonio, TX (US); Won Jae KIM, San Antonio, TX (US); Ji-Yeon JUNG, San Antonio, TX (US)

(73) Assignee: THE BOARD OF REGENTS OF THE UNIVERSITY OF TEXAS SYSTEM, Austin, TX (US)

(21) Appl. No.: 15/580,945

(22) PCT Filed: Jun. 10, 2016
(86) PCT No.: PCT/US16/36875

§ 371 Date: Dec. 8, 2017

Related U.S. Application Data
(60) Provisional application No. 62/173,837, filed on Jun. 10, 2015.

(51) Int. Cl. C12Q 1/6886; A61K 33/24; A61K 31/282; A61K 31/4706; A61K 31/365; A61K 31/52; A61K 31/5377; A61K 31/4418; A61K 31/7048; A61K 31/4184; A61K 38/08; A61K 31/517; A61K 31/439; A61K 31/366; A61K 31/12; A61P 35/00; G01N 33/574

(52) U.S. Cl. CPC C12Q 1/6886; G01N 2333/705; A61K 31/282; A61K 31/4706; A61K 31/365; A61K 31/52; A61K 31/5377; A61K 31/4418; A61K 31/7048; A61K 31/4184; A61K 38/08; A61K 31/517; A61K 31/439; A61K 31/366; A61K 31/12; A61P 35/00; G01N 33/57492; C12Q 2600/106; C12Q 2600/158; A61K 33/24

(57) ABSTRACT

Certain embodiments are directed to methods and composition for detecting the level of GFRα1 in cancers and treating same.

This application claims priority to U.S. Provisional Patent Application Ser. No. 62/173,837 filed Jun. 10, 2015, which is incorporated herein by reference in its entirety.

### BACKGROUND

Chemotherapy is a widely used therapeutic regimen for cancer treatment. Recent progress in chemotherapy has significantly increased its efficacy, yet the development of chemoresistance following prolonged and repeated treatment remains a major drawback.

There remains a need for additional methods for identifying chemoresistance as well as methods for treating chemoresistant cancers.

### SUMMARY

GFRα1 is a novel receptor that contributes to cisplatin-induced chemoresistance and metastasis. In certain aspect GFRα1 is elevated in chemoresistant cancer such as osteosarcoma.

Certain embodiments are directed to molecules that are differentially expressed in cells that have become chemoresistant, such as GFRα1. Accordingly, one aspect is directed to methods of determining if a subject or a subject's cancer has become or is at risk of becoming chemoresistant. The methods can include measuring the level of a biomarker in a sample or in one or more cancer cells, wherein an increased level of the biomarker indicates that the subject's cancer is or will become chemoresistant. In certain aspects the biomarker is a GFRα1 nucleic acid or polypeptide. In certain aspects the cancer is resistant to a platinum based chemotherapeutic. The platinum based chemotherapeutic can be carboplatin, cisplatin, oxaliplatin, BBR3464, or satraplatin. In a specific embodiment, the platinum based therapeutic is cisplatin.

In a further aspect the cancer is bone, pancreatic, kidney, stomach, brain, colon, skin, lung, bladder, prostate, uterine, cervical, breast or ovarian cancer. In certain aspects the cancer is bone cancer, e.g., osteosarcoma.

The level of a biomarker can be elevated, increased, or higher than a reference or control. In one embodiment, the control is a sample from a non-cancerous tissue.

Certain embodiments are directed to methods of treating a subject having cancer. The biomarker can be used to identify a cancer that does not have elevated levels of GFRα1 and are sensitive to platinum based chemotherapies. GFRα1 levels can also be used to identify those cancers that are resistant and as such dictate a non-platinum based chemotherapy, or a more aggressive therapy. In certain aspects the GFRα1 biomarker is a GFRα1 nucleic acid or polypeptide.

In another embodiment, the invention provides a method for determining the prognosis of a subject having cancer comprising measuring the level of GFRα1, wherein an increased level of GFRα1 is indicative of poor prognosis.

A “biological sample” in terms of the invention means a sample of biological tissue or fluid. Examples of biological samples are sections of tissues, blood, blood fractions, plasma, serum, urine or samples from other peripheral sources. A biological sample may be provided by removing a sample from a subject, but can also be provided by using a previously isolated sample. For example, a tissue sample can be removed from a subject suspected of having a disease by conventional biopsy techniques. In a preferred embodiment, a blood sample is taken from the subject. In one embodiment, the blood or tissue sample is obtained from the subject prior to, during, and/or after initiation of radiotherapy, chemotherapy, or other therapeutic treatment. According to the invention, the biological sample preferably is a tissue sample comprising suspected cancer cells.

“Polynucleotide,” synonymously referred to as “nucleic acid molecule” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, double-stranded, or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

“Polypeptide” refers to any peptide or protein comprising amino acids joined by peptide bonds or modified peptide bonds. “Polypeptide” refers to short chains, including peptides, oligopeptides or oligomers, and to longer chains, including proteins. Polypeptides may contain amino acids other than the 20 gene-encoded amino acids. “Polypeptides” include amino acid sequences modified either by natural processes, such as post-translational processing, or by chemical modification or other synthetic techniques well known in the art.

“Antibody” refers to all isotypes of immunoglobulins (IgG, IgA, IgE, IgM, IgD, and IgY) including various monomeric and polymeric forms of each isotype, unless otherwise specified.

“Functional fragments” of such antibodies comprise portions of intact antibodies that retain antigen-binding specificity of the parent antibody molecule. For example, functional fragments can comprise at least the CDRs of either the heavy chain or light chain variable region. Functional fragments can also comprise the heavy chain or light chain variable region, or sequences that are substantially similar to the heavy or light chain variable region. Further suitable functional fragments include, without limitation, antibodies with multiple epitope specificity, bispecific antibodies, diabodies, and single-chain molecules, as well as Fab, F(ab′)2, Fd, Fabc, and Fv molecules, single chain (Sc) antibodies (also called ScFv), individual antibody light chains, individual antibody heavy chains, chimeric fusions between antibody chains and other molecules, heavy chain monomers or dimers, light chain monomers or dimers, dimers consisting of one heavy and one light chain, and the like. All antibody isotypes can be used to produce functional fragments of the antibodies herein. Functional fragments can be recombinantly or synthetically produced, with natural or unnatural nucleic acid or amino acid molecules.

The term “cancer” includes, but is not limited to, solid tumors, such as cancers of the bone, breast, colon, lung, brain, reproductive organs, cervix, digestive tract, urinary tract, eye, liver, skin, head and neck, thyroid, parathyroid, and their distant metastases. The term also includes lymphomas, sarcomas (e.g., osteosarcomas), and leukemias.

As used herein, the term “level of expression” refers to the measurable expression level of a given polypeptide or nucleic acid molecule. The term “differentially expressed” or “differential expression” refers to an increase or decrease in the measurable expression level of a given polypeptide or nucleic acid. Absolute quantification of the level of expression of a polypeptide or nucleic acid may be accomplished by comparing the level to that of a reference or control. The control can be an average amount of the molecule in a statistically significant number of samples, or can be compared to a the level of the molecule in a non-cancerous sample.

In preferred embodiments of the invention, a cancer having elevated levels of GFRα1 is resistant to is a platinum based therapeutic. In certain aspects the platinum based therapeutic is carboplatin, cisplatin, oxaliplatin, BBR3464, satraplatin. In certain aspects, the chemotherapeutic agent is cisplatin.

As used herein, the term,“chemoresistant” refers to subjects who fail to respond to the action of one or more chemotherapeutic agents. Most subjects are not chemoresistant at the beginning of treatment but may become so after a period of treatment. In specific embodiments, chemoresistant cancers are resistant to platinum based therapeutics. In a particular embodiment, the subjects are chemoresistant to cisplatin.

The biomarkers of the invention can be nucleic acid or polypeptide biomarkers. In a preferred embodiment, the biomarkers are polypeptides. The instant invention is based on the finding that certain molecules are differentially expressed in cells that have become, or are becoming chemoresistant. In order to determine if a cell is chemoresistant, or at risk of becoming chemoresistant, the instant invention provides methods for determining the level of the identified biomarkers in a biological sample. Certain embodiments provide methods and compositions for determining the amount of a protein or nucleic acid biomarker of the invention in a biological sample.

The biomarker can be bound or form a complex with a GFRα1 binding reagent, such as an antibody or a nucleic acid probe. The antibodies or functional fragments thereof of the disclosed subject matter can be generated from any species. The probes, antibodies or functional fragments thereof described herein can be labeled or otherwise conjugated to various chemical or biomolecule moieties, for example, for therapeutic or diagnostic or detection or treatment applications. The moieties can be detectable labels, for example, fluorescent labels, radiolabels, biotin, and the like, which are known in the art.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

“Effective amount” and “therapeutically effective amount” are used interchangeably herein, and refer to an amount of an antibody or functional fragment thereof, as described herein, effective to achieve a particular biological or therapeutic result such as, but not limited to, the biological or therapeutic results disclosed herein. A therapeutically effective amount of the antibody or antigen-binding fragment thereof may vary according to factors such as the disease state, age, sex, and weight of the individual, and the ability of the antibody or functional fragment thereof to elicit a desired response in the individual. Such results may include, but are not limited to, the treatment of cancer, as determined by any means suitable in the art.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

“Prognosis” refers to a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis and/or cancer progression in a patient susceptible to or diagnosed with a cancer. Prognosis also includes prediction of favorable responses to cancer treatments, such as a conventional cancer therapy. A good or bad prognosis may, for example, be assessed in terms of patient survival, likelihood of disease recurrence, disease metastasis, or disease progression (patient survival, disease recurrence and metastasis may for example be assessed in relation to a defined time point, e.g. at a given number of years after cancer surgery or after initial diagnosis). In one embodiment, a good or had prognosis may be assessed in terms of overall survival, disease-free survival or progression-free survival.

In one embodiment, the biomarker level is compared to a reference level representing the same biomarker. In certain aspects, the reference level may be a reference level of expression from non-cancerous tissue from the same subject. Alternatively, reference level may be a reference level of expression from a different subject or group of subjects. For example, the reference level of expression may be an expression level obtained from tissue of a subject or group of subjects without cancer, or an expression level obtained from non-cancerous tissue of a subject or group of subjects with cancer. The reference level may be a single value or may be a range of values. The reference level of expression can be determined using any method known to those of ordinary skill in the art. In some embodiments, the reference level is an average level of expression determined from a cohort of subjects with cancer. The reference level may also be depicted graphically as an area on a graph.

The reference level may comprise data obtained at the same time (e.g., in the same hybridization experiment) as the patient's individual data, or may be a stored value or set of values e.g. stored on a computer, or on computer-readable media. If the latter is used, new patient data for the selected marker(s), obtained from initial or follow-up samples, can be compared to the stored data for the same marker(s) without the need for additional control experiments.

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Cisplatin induces GFRα1 expression in osteosarcoma cells. (a-c) Immunoblot analysis of osteosarcoma cell lysates with antibodies specific for GFRα1 and β-actin. (a) MG-63 and U-2 OS cells were treated with doxorubicin (5 μM), cisplatin (20 μM), or methotrexate (1 mM) for 24 hr. Immunoblot analysis of GFRα1 (left) and quantification of GFRα1 expression (right) after treatment of chemotherapeutic agents. (b) MG-63 and U-2 OS cells were treated with different concentrations of cisplatin for 24 hr. (c) MG-63 and U-2 OS cells were treated with cisplatin (20 μM) and collected at the indicated time. (d and e) Quantitative real-time PCR of GFRα1 mRNA expression after cisplatin treatment. Representative images (top) and quantitative analysis (bottom) of GFRα1 mRNA expression. The values are presented as a mean±s.d.m (n=3). ** denotes p<0.05 by t-test. (d) MG-63 and U-2 OS cells were treated with different concentrations of cisplatin for 24 hr. (e) MG-63 and U-2 OS cells were treated with cisplatin (20 μM) and collected at the indicated time.

FIG. 2. GFRα1 suppresses the chemosensitivity of osteosarcoma cells induced by cisplatin. (a) Generation of GFRα1-deficient osteosarcoma cell lines with GFRα1 shRNA. Both MG-63 and U-2 OS cells were transfected with control or GFRα1 shRNA. Immunoblot analysis of control and GFRα1-deficient stable osteosarcoma cell lysates with antibodies specific for GFRα1 and β-actin. (b) Cell viability of GFRα1-deficient osteosarcoma cells after cisplatin treatment. Control or GFRα1-deficient cells were cultured and treated with different concentrations of cisplatin for 24 hr. Cell viability was measured using the WST-1 assay. The values are represented as a mean±s.d.m. (n=3). ** denotes p<0.05 by t-test. (c) Apoptotic response of GFRα1-deficient osteosarcoma cells after cisplatin treatment. Control and GFRα1-deficient cells were cultured and treated with different concentrations of cisplatin for 24 hr. Apoptotic cells by FITC-Annexin V staining (top) and viable cells by PI staining (bottom) were analyzed through flow cytometry. The values are represented as a mean±s.d.m. (n=3). ** denotes p<0.05 by t-test. (d) Immunoblot analysis of control and GFRα1-deficient MG-63 cell lysates with antibodies specific for apoptotic proteins, cleaved PARP and cleaved Caspase-3. Control and GFRα1-deficient MG-63 cells were treated with cisplatin (20 μM) for 24 hr and cells were collected. (e) Relative Caspase-3 activity in control and GFRα1-deficient MG-63 cells after cisplatin treatment. Control and GFRα1-deficient MG-63 cells were treated with cisplatin (20 μM) for 24 hr in the presence or absence of ZVAD-FMK (50 μM). The values are represented as a mean±s.d.m. (n=3). ** denotes p<0.05 by t-test. (f) Generation of GFRα1-overexpressing osteosarcoma cell lines with GFRα1 expression vector. Both MG-63 and U-2 OS cells were transfected with control or human GFRα1 expression vector. Immunoblot analysis of control and GFRα1-overexpressing stable osteosarcoma cell lysates with antibodies specific for GFRα1 and β-actin. (g) Cell viability of GFRα1-overexpressing osteosarcoma cells after cisplatin treatment. Control and GFRα1-overexpressing cells were cultured and treated with different concentrations of cisplatin for 24 hr. Cell viability was measured using the WST-1 assay. The values are represented as a mean±s.d.m. (n=3). ** denotes p<0.05 by t-test. (h) Apoptotic response of GFRα1-overexpressing osteosarcoma cells after cisplatin treatment. Control and GFRα1-38 overexpressing cells were cultured and treated with different concentrations of cisplatin for 24 hr. Apoptotic cells by FITC-Annexin V staining (top) and viable cells by PI staining (bottom) were analyzed through flow cytometry. The values are represented as a mean±s.d.m. (n=3). ** denotes p<0.05 by t-test.

FIG. 3. GFRα1-induced autophagy enhances the chemoresistance of osteosarcoma cells induced by cisplatin. (a) Control and GFRα1-deficient MG-63 cells were transiently transfected with a mRFP-GFP tandem fluorescent-tagged LC3 (mRFP-GFP-LC3) vector and then treated with cisplatin (20μM) for 24 hr. Left, representative images of RFP-LC3 and GFP-LC3 puncta. Scale bar, 20 μm. Right, quantitative analysis of the number of yellow puncta and the number of RFP-LC3 puncta in the combined images. The values are represented as a mean±s.d.m. (n=3). ** denotes p<0.05 by t-test. (b) Control and GFRα1-deficient MG-63 cells were treated with cisplatin (20 μM) for 24 hr and then stained with acridine orange. Top, representative images of cells stained with acridine orange. Scale bar, 20 μm. Bottom, quantitative analysis of the number of AVOs. The values are represented as a mean±s.d.m. (n=3). ** denotes p<0.05 by t-test. (c) Control and GFRα1-deficient MG-63 cells were treated with cisplatin (20 μM) for 24 hr and then analyzed by TEM. Top, representative images of autophagic vacuoles (yellow arrows) detected after cisplatin treatment in control MG-63 cells. Scale bar, 100 nm. Bottom, quantitative analysis of the number of autophagic vacuoles. The values are represented as a mean±s.d.m. (n=3). ** denotes p<0.05 by t-test. (d) Immunoblot analysis of control and GFRα1-overexpressing osteosarcoma cell lysates with antibodies specific for GFRα1, LC3 and β-actin. GFRα1-overexpressing MG-63 cells were treated with DMSO, 3-MA (10 μM) or Baf (100 nM). (e) Quantitative analysis of the number of yellow puncta and the number of RFP-LC3 puncta in control and GFRα1-overexpressing MG-63 cells. Control and GFRα1-overexpressing MG-63 cells were transiently transfected with an RFP-GFP tandem fluorescent-tagged LC3 (mRFP-GFP-LC3) and then treated with cisplatin (20 μM) for 24 hr. (f) Control and GFRα1-overexpressing MG-63 cells were pretreated with 3-MA (10μM) for 2 hr before mRFP-GFR-LC3 transfection and were then incubated for 24 hr. (g) Control and GFRα1-overexpressing MG-63 cells were treated with cisplatin (20 μM) for 24 hr and then stained with acridine orange. Top, representative images of cells stained with acridine orange. Scale bar, 20 μm. Bottom, quantitative analysis of the number of AVOs. The values are represented as a mean±s.d.m. (n=3). ** denotes p<0.05 by t-test. (h) Control and GFRα1-overexpressing MG-63 cells were treated with cisplatin (20 μM) for 24 hr and then analyzed by TEM. Top, representative images of autophagic vacuoles detected after cisplatin treatment in control and GFRα1-overexpressing MG-63 cells. Scale bar, 100 nm. Bottom, quantitative analysis of the number of autophagic vacuoles. The values are represented as a mean±s.d.m. (n=3). ** denotes p<0.05 by t-test. (i) Cell viability of GFRα1-overexpressing osteosarcoma cells after cisplatin treatment in the presence of autophagy inhibitors. Control or GFRα1-overexpressing MG-63 cells were cultured with DMSO, 3-MA or Baf, and then treated with cisplatin (20 μM) for 24 hr. Cell viability was measured using the WST-1 assay. The values are represented as a mean±s.d.m. (n=3). ** denotes p<0.05 by t-test. (j) Cell viability of GFRα1-overexpressing osteosarcoma cells after cisplatin treatment in the presence of Beclin 1 (20 nM) or HMGB1 (20 nM) 40 siRNA. Control or GFRα1-overexpressing MG-63 cells were cultured with Beclin 1 or HMGB1 siRNA for 48 hr and then treated with cisplatin (20 μM) for 24 hr. Cell viability was measured using the WST-1 assay. The values are represented as a mean±s.d.m. (n=3). ** denotes p<0.05 by t-test. (k) The effect of GFRα1 deficiency or overexpression on colony formation in the presence of cisplatin (20 μM). Equal number (1×10⁴ cells) of control (control shRNA), GFRα1-deficient (GFRα1 shRNA), control (empty vector), and GFRα1-overexpressing (GFRα1 expressing vector) MG-63 cells were plated. All of cell lines were incubated with cisplatin (20 μM) for 10 days. Colonies were stained by crystal violet for visualization. Left, representative images of colony formation. Right, quantitative analysis of the number of colonies and plating efficiency. The values are represented as a mean±s.d.m. (n=3). ** denotes p<0.05 by t-test. (1) The effect of autophagy inhibitors on the colony formation of GFRα1-overexpressing cells in the presence of cisplatin (20 μM). Control and GFRα1-overexpressing clone #4 (See above FIG. 3j were plated and treated with 3-MA (10 μM), Baf (100 nM), or CQ (30 μg/ml), respectively. All of cell lines were incubated with cisplatin for 10 days. All of plates were scanned by scanner and numbers of colonies were quantified by using image-J software. Left, representative images of colony formation. Right, quantitative analysis of the number of colonies and survival fraction. The values are represented as a mean±s.d.m. (n=3). ** denotes p<0.05 by t-test. (m) MG-63 cells were pretreated with 3-MA (10 mM) for 2 hr before mRFP-GFR-LC3 transfection and were then treated with cisplatin (20 μM) for 24 hr. (n) Generation of BECN1-deficient osteosarcoma cell line with BECN1 shRNA. MG-63 cells were transfected with control or BECN1 shRNA. Immunoblot analysis of control and BECN1-deficient stable osteosarcoma cell lysates with antibodies specific for BECN1 and ACTB. (o) Control and BECN1-deficient MG-63 cells were transiently transfected with a mRFP-GFP tandem fluorescent-tagged LC3 (mRFP-GFP-LC3) vector and then treated with cisplatin (20 04) for 24 hr. (p) Control and GFRα1-overexpressing MG-63 cells were pretreated with 3-MA (10 mM) for 2 hr before mRFP-GFR-LC3 transfection and were then incubated for 24 hr. (q) Control, GFRα1-overexpressing, and BECN1-deficient/GFRα1-overexpressing MG-63 cells were transiently transfected with an RFP-GFP tandem fluorescent-tagged LC3 (mRFP-GFP-LC3) and then treated with cisplatin (20 μM) for 24 hr.

FIG. 4. GFRα1 regulates autophagy by Src/AMPK signaling in osteosarcoma cells. (a-b) Immunoblot analysis of osteosarcoma cell lysates with antibodies specific for GFRα1, p-Src, Src, p-AMPK, AMPK, p-mTOR, mTOR, p-p70 S6Kinase, Beclin 1, HMGB1, LC3, and β-actin. (a) MG-63 and cells were treated with cisplatin (20 μM) for 24 hr. (b) Control and GFRα1-deficient MG-63 cells were treated with cisplatin (20 μM) for 24 hr. (c-e) Immunoblot analysis of osteosarcoma cell lysates with antibodies specific for GFRα1, p-Src, Src, p-AMPK, AMPK, LC3, and β-actin. (c) Control and Src-deficient MG-63 cells were treated with cisplatin (20 μM) for 24 hr. (d) MG-63 cells were cultured in the presence or absence of PP1 (5 μM) and then treated with cisplatin (20 μM) for 24 hr. (e) MG-63 cells were cultured in the presence or absence of Compound C and then treated with cisplatin (20 μM) for 24 hr. (f) Cell viability of MG-63 cells after cisplatin treatment in the presence of PP1. MG-63 cells were cultured with DMSO or PP1 (2, 5 μM) and then treated with cisplatin (20 μM) for 24 hr. Cell viability was measured using the WST-1 assay. The values are represented as a mean±s.d.m. (n=3). ** denotes p<0.05 by t-test. (g) Cell viability of MG-63 cells after cisplatin treatment in the presence of Compound C. MG-63 cells were cultured with DMSO or Compound C (5, 10 μM) and then treated with cisplatin (20 μM) for 24 hr. Cell viability was measured using the WST-1 assay. The values are represented as a mean±s.d.m. (n=3). ** denotes p<0.05 by t-test.

FIG. 5. GFRα1-mediated autophagy promotes chemoresistance and tumor growth in vivo. (a) BALB/c nude mice (n =40) were injected with MG-63 cells that were transfected with either control or GFRα1 expressing vector. Tumor volume was measured every 3 or 4 days. Data are shown as mean±SEM. ** p<0.05. (b) BALB/c nude mice (n=20) were injected with MG-63 cells that were transfected with either control or GFRα1 shRNA. Tumor volume was measured every 3 or 4 days. Data are shown as mean±SEM. ** p<0.05. (c) Tumors that were generated from mice injected with MG-63 cells containing GFRα1 expressing vector were directly injected with PBS, CQ, cisplatin, or cisplatin+CQ. Tumor volume was measured every 4 days. Data are shown as mean±SEM. ** p<0.05 vs PBS, CQ, or cisplatin-treated tumors. (d) Left, representative images of immunofluorescence staining of HMGB1 in a section from tumors generated from mice injected with MG-63 cells containing GFRα1 expressing vector and then treated with PBS, CQ, cisplatin, or cisplatin+CQ. Right, quantitative analysis of percentage of cytoplasmic HMGB1-positive cells in HMGB1-positive tumors. The values are represented as a mean±s.d.m. (n=3). ** denotes p<0.05 by t-test. (e) Left, representative images of TUNEL-positive cells in a section from tumors generated from mice injected with MG-63 cells containing GFRα1 expressing vector and then treated with PBS, CQ, cisplatin, or cisplatin+CQ. Right, quantitative analysis of percentage of TUNEL-positive cells in HMGB1-positive tumors. The values are represented as a mean±s.d.m. (n=3). ** denotes p<0.05 by t-test. (0 Survival rate of mice injected with MG-63 cells containing GFRα1 expressing vector and then treated with either cisplatin or cisplatin+CQ. (g) Representative images of immunohistochemical staining of GFRα1 in a section from human osteosarcoma patient before and after cisplatin treatment. (h) Representative images of immunofluorescence staining of GFRα1 and HMGB1 in a section from human osteosarcoma patient before and after cisplatin treatment.

FIG. 6. Cell viability of GFRα1-deficient osteosarcoma cells after treatment of chemotherapeutic agents. MG-63 and U-2 OS cells were transfected with either control siRNA or GFRα1 siRNA for 48 h and then treated with different concentrations of doxorubicin, cisplatin, or methotrexate for 24 h. Cell viability was measured using the WST-1 assay. The values are represented as a mean±s.d.m. (n=3). ** denotes P<0.05 by t-test. (a) MG-63 cells. (b) U-2 OS cells.

FIG. 7. GFRα1-mediated chemoresistance of osteosarcoma cells is independent of GDNF. (a) Quantitative real-time PCR of GDNF mRNA expression after cisplatin treatment. MG-63 and U-2 OS cells were treated with different concentrations of cisplatin for 24 h. (b-d) Cell viability was measured using the WST-1 assay. The values are represented as a mean±s.d.m. (n=3). (b) MG-63 cells were treated with PBS or GDNF (50 ng/ml) for 24 h. (c) Control and GFRα1-overexpressing cells were cultured and treated with PBS or GDNF for 24 h. (d) Control and GFRα1-overexpressing cells were treated and cultured with PBS only, Cisplatin, or Cisplatin+GDNF (50 ng/ml) for 24 h, respectively. (f) Representative images of RFP-LC3 and GFP-LC3 puncta. Scale bar, 20 μm. Control and GFRα1-overexpressing MG-63 cells were transiently transfected with an RFP-GFP tandem fluorescent-tagged LC3 (RFP-GFP-LC3) and then treated with PBS (top two panels) or cisplatin (20 μM; bottom two panes) for 24 h. Control and GFRα1-overexpressing MG-63 cells were also transiently transfected with an RFP-GFP tandem fluorescent-tagged LC3 vector (RFP-GFP-LC3) and then treated with PBS or GDNF for 24 h in the absence (third and fourth panels) or presence (fifth and sixth) of 3-MA. (f) Quantitative analysis of the number of yellow puncta and the number of RFP-LC3 puncta in the combined images of Control and GFRα1-overexpressing MG-63 cells treated with GDNF. The values are represented as a mean±s.d.m. (n=3). ** denotes P<0.05 by t-test.

FIG. 8. Effect of GFRα1 on cisplatin-induced apoptosis. (a) Apoptotic cells were counted in control or GFRα1-deficient MG-63 cells by flow cytometry 24 h after cisplatin treatment. (b) Under the same conditions, apoptotic cells were counted in control or GFRα1-overexpressing MG-63 cells. (c) Apoptotic cells were counted in control and GFRα1-deficient U-2 OS cells by flow cytometry 24 h after cisplatin treatment. (b) Under the same conditions, apoptotic cells were counted in control or GFRα1-overexpressing U-2 OS cells.

FIG. 9. Acridine orange staining of GFRα1-deficient and GFRα1-overexpressing U-2 OS cells after cisplatin treatment. (a) Control and GFRα1-deficient U-2 OS cells were treated with cisplatin (20 μM) for 24 h and then stained with acridine orange (0.5 mg/ml) for 15 min. Top, representative images of cells stained with acridine orange. Scale bar, 20 μm. Bottom, quantitative analysis of the number of AVOs. The values are represented as a mean±s.d.m. (n=3). ** denotes P<0.05 by t-test. (b) Control and GFRα1-overexpressing U-2 OS cells were treated with cisplatin (20 μM) for 24 h and then stained with acridine orange. Top, representative images of cells stained with acridine orange. Scale bar, 20 μm. Bottom, quantitative analysis of the number of AVOs. The values are represented as a mean±s.d.m. (n=3). ** denotes P<0.05 by t-test.

FIG. 10. GFRα1-mediated chemoresistance of osteosarcoma cells is independent of APE and RET signaling. (a) Immunoblot analysis of MG-63 cell lysates with antibodies specific for APE, RET and β-actin. MG-63 and U-2 OS cells were treated with different concentrations of cisplatin for 24 h. The cell lysates of GDNF-treated MIA PaCa-2 were used as positive controls for APE and RET expression.

FIG. 11. GFRα1 and NFκB p50 expression in cisplatin-resistant MG-63 cells. (a) Representative images of colony formation in MG-63 resistant cell clones after cisplatin treatment for 10 days. (b) Quantitative analysis of the number of colonies. The values are represented as a mean±s.d.m. (n=3). ** denotes P<0.05 by t-test. (c) Quantitative real-time PCR of NFκB p50 mRNA expression in MG-63 (control) or MG-63 resistant cell clones 24 h after cisplatin treatment. The values are presented as a mean±s.d.m (n=3). ** denotes P<0.05 by t-test. (d) Quantitative real-time PCR of GFRα1 mRNA expression in MG-63 (control) or MG-63 resistant cell clones 24 h after cisplatin treatment. The values are presented as a mean±s.d.m (n=3). ** denotes P<0.05 by t-test. (e) Quantitative real-time PCR of NFκB p50 mRNA expression in MG-63 cells transiently transfected with either control siRNA or NFκB p50 siRNA for 48 h and then treated with cisplatin for 24 h. The values are presented as a mean±s.d.m (n=3). ** denotes P<0.05 by t-test. (I) Quantitative real-time PCR of GFRα1 mRNA expression in MG-63 cells transiently transfected with either control siRNA or NFκB p50 siRNA for 48 h and then treated with cisplatin for 24 h. The values are presented as a mean±s.d.m (n=3). ** denotes P<0.05 by t-test.

FIG. 12. Cellular transformation of NIH3T3 cells by GFRα1. NIH3T3 cells (1×10⁶) were cocultured with empty vector-expressing NIH3T3 cells (1×10³) or GFRα1-expressing NIH3T3 cells (1×10³). (a-b) Representative images of phase-contrast microscopy (c, d) Representative images of crystal violet staining.

FIG. 13. (a) Representative images of immunofluorescence staining of GFRα1 in a section from tumors generated from mice injected with MG-63 cells containing GFRα1 expressing vector and then treated with PBS, CQ, cisplatin, or cisplatin+CQ. (b) Survival rate of mice injected with MG-63 cells containing GFRα1 expressing vector and then treated with PBS, CQ, cisplatin, or cisplatin+CQ.

FIG. 14. Schematic diagram for GFRα1-mediated autophagy in cisplatin-induced chemoresistance of osteosarcoma. Cisplatin treatment activates NFκB signaling, by either direct or indirect manner, and then activated NFκB promotes the expression of GFRα1 by binding the promoter region of GFRα1 gene (27). It is possible that cisplatin treatment could induce ligand(s) other than GDNF that can bind to GFRα1. Cisplatin-induced expression/activation of GFRα1 then phosphorylates its downstream kinase Src and subsequently activates AMPK/mTOR-dependent autophagy signaling. Src inhibitor PP1 and AMPK inhibitor Compound C can block cisplatin-mediated autophagy. Inhibitors of autophagy, 3-MA, Baf, or CQ can also block cisplatin-mediated autophagy. The regulation of GFRα1 expression/activation by cisplatin can contribute to the development of chemoresistance in osteosarcoma through Src/AMPK/mTOR-mediated autophagy signaling, which is independend of GDNF, RET, or APE signaling. 3-MA:3-methyladenine; Baf:bafilomycin A1; CQ: chloroquine.

FIG. 15. GFRα1 levels in cancers of the cervix, breast, brain, colon, and skin.

### DESCRIPTION

Chemotherapy is a widely used therapeutic regimen for cancer treatment. Recent progress in chemotherapy has significantly increased its efficacy, yet the development of chemoresistance following prolonged and repeated treatment remains a major drawback. It is shown that glial cell line-derived neurotrophic factor (GDNF) receptor α1, or GFRα1 (GenBank accession numbers NM_001145453.1 (GI 224282177), NM_145793.3 (GI 224282172), or NM_005264.4 (GI 224282171), each of which is incorporated herein as of the filing date of the current application), contributes to cisplatin-induced chemoresistance in cancers such as osteosarcoma. The level of GFRα1 mRNA and protein expression were increased in human osteosarcoma cells after cisplatin treatment. Knockdown of GFRα1 expression significantly enhanced cisplatin-induced apoptotic cell death, while overexpression of GFRα1 reduced it. Additionally, GFRα1 expression significantly increased cisplatin-induced autophagy, and inhibition of autophagy in GFRα1-overexpressing cells restored the chemosensitivity to cisplatin. GFRα1 induced Src phosphorylation and then AMP-activated protein kinase (AMPK) phosphorylation, in cisplatin-treated osteosarcoma cells, resulted in increased autophagy. Inhibition of Src or AMPK activation repressed cisplatin-induced autophagy and further reduced cell viability. GFRα1 expression promoted tumor formation and growth in mouse xenograft models and injection of chloroquine, an autophagy inhibitor, in GFRα1-overexpressing tumors inhibited autophagy and significantly reduced tumor growth. Moreover, treatment period and metastatic status in human patients treated with cisplatin is associated with increased expression of GFRα1. These findings suggest that autophagy by GFRα1/Src/AMPK axis is a promising target for novel therapeutic approaches in overcoming cisplatin-induced chemoresistance in cancers such as osteosarcoma.

Osteosarcoma is the predominant form of primary bone cancer. It is a highly malignant tumor that mostly arises in childhood and adolescence (1). Use of chemotherapy along with surgery has raised the long-term survival rate of osteosarcoma patients to approximately 70% (2,3). Doxorubicin, cisplatin, and methotrexate are commonly used chemotherapeutics active against osteosarcoma. In particular, cisplatin is the most widely used platinum-based anticancer drug for solid tumors including osteosarcoma (4). It interacts with nucleophilic N7 sites of purine bases in DNA to induce DNA damage that leads to inhibition of tumor cell division and initiation of apoptosis, or programmed cell death (5). Although this treatment strategy is highly effective, it is often limited by acquired or intrinsic resistance of cancer cells to the drug (6). Thus, understanding the molecular mechanisms which lead to chemoresistance is essential to developing more effective treatments against osteosarcoma.

Autophagy is a critical process by which cells self-digest and recycle inessential or ineffectual cellular components to maintain homeostasis, especially under conditions of metabolic stress (7,8). During the initial stages of autophagy, cellular proteins, organelles and cytoplasm are sequestered and engulfed by autophagosomes. The autophagosomes then fuse with lysosomes to form the autolysosomes, where the sequestered proteins and organelles are digested by lysosomal hydrolases (9,10). Autophagy can play a role in cell death as an alternative cell death mechanism known as programmed cell death type II, particularly within apoptosis-deficient cells and through this mechanism it can function in tumor suppression (11-13). Paradoxically, autophagy has also been established as a cell survival mechanism that is induced by environmental stresses including nutrient deficiency, chemotherapy, radiation, and hypoxia (11,12,14). Studies have shown that induction of autophagy for cell survival can confer resistance to anticancer therapies in some cancers (15-19). However, the relative contribution of autophagy to either apoptotic cell death as a tumor suppressive mechanism or cell survival as a survival-promoting mechanism during anticancer treatment remains mostly unresolved.

Glycosylphosphatidylinositol-linked glial cell line-derived neurotrophic factor (GDNF) receptor α, or GFRα, is a co-receptor that recognizes the GDNF family of ligands, which includes GDNF, neuturin, artemin, and persephin (20,21). Four different GFRα receptors have been identified. Each GFRα specifically recognizes its own GDNF ligand. Binding of GFRα with its ligand activates protein tyrosine kinase RET and subsequently activates Src, a member of the Src family of cytoplasmic tyrosine kinases (20). GDNF/GFRα signaling regulates the development and maintenance of the nervous system by protecting and promoting survival of dopaminergic neurons and thereby it has potential as a therapeutic target for neurodegenerative diseases such as Parkinson's disease and Alzheimer's disease (22,23). GFRα1 specifically recognizes GDNF and it has been implicated in the regulation of neuronal cell survival and differentiation. Studies also have indicated that GFRα1 has a role in the progression of human cancers such as breast cancer and pancreatic cancer by promoting migration and invasion (24-27), although the exact mechanism for oncogenesis remains unclear. Here, a novel mechanism is described in which GFRα1 contributes to the development of cisplatin-induced chemoresistance in osteosarcoma by facilitating autophagy via Src/AMP-activated protein kinase (AMPK) signaling.

Chemoresistance arises in various ways mediated by drug export transporters, DNA repair mechanisms, cancer stem cells, resistance to apoptosis, self-sufficiency for growth factor signaling, angiogenic switch, and immunological pathways (16). Autophagy can contribute to increased acquisition of chemoresistance in cancer; however, it also contributes to the inhibition of chemoresistance in some types of cancer (16,43). These conflicting findings suggest that the role autophagy plays in chemoresistance may depend on the facilitating signaling mechanisms that are differentially regulated based on cancer type and/or the chemotherapy strategy used. Therefore, elucidation of autophagic signaling mechanisms with regards to specific type of cancers and therapies is required to develop more effective chemotherapeutic combinations for cancer treatment. The study described herein provides the first evidence that the chemotherapy drug cisplatin induces expression of GFRα1 receptor which inhibits cisplatin-mediated apoptosis and triggers autophagy for cell survival in vitro and in vivo, thereby promoting chemoresistance. It was found that tumors from 4 of 9 patients who had undergone extended cisplatin treatment expressed both GFRα1 and HMGB1 and metastasized to the lungs (Table 1 and Table 3). The clinical data substantiates the in vivo results seen in xenograft mouse models, which implies similar results could have been obtained with an orthotopic model.

GFRα1 functions by binding to GDNF and when they are in complex GDNF/GFRα1 signaling can proceed through either activation of proto-oncogene SRC or through activation of RET which in turn activates SRC (20, 21). GDNF promotes resistance to the cytotoxic effects of all-trans-retinoic acid in neuroblastoma cells and it contributes to the chemoresistance to 1,3-bis(2-chloroethyl)-1-nitrosourea in glioblastoma cells by regulating Akt (Protein kinase B) and JNK (c-Jun NH(2)-terminal kinase) survival signaling (44,45). GDNF significantly increases the survival of hair cells located in the cochlea of the inner ear after cisplatin treatment both in vivo and in vitro, indicating GDNF can be protective against cisplatin-induced ototoxicity (46,47). Furthermore, GDNF is important to spermatogonial stem cell (SSC) proliferation and self-renewal by regulating transcription factors Bc16b, Erm, and Lhx1 through SFK signaling (48). With this regard, GDNF expression is increased along the basal Sertoli cell membrane, the physical location of the SSC niche, throughout the recovery period after repeated cisplatin treatment, indicating that redistribution of GDNF expression after cisplatin treatment is closely correlated with the expansion of the SSC population (49,50).

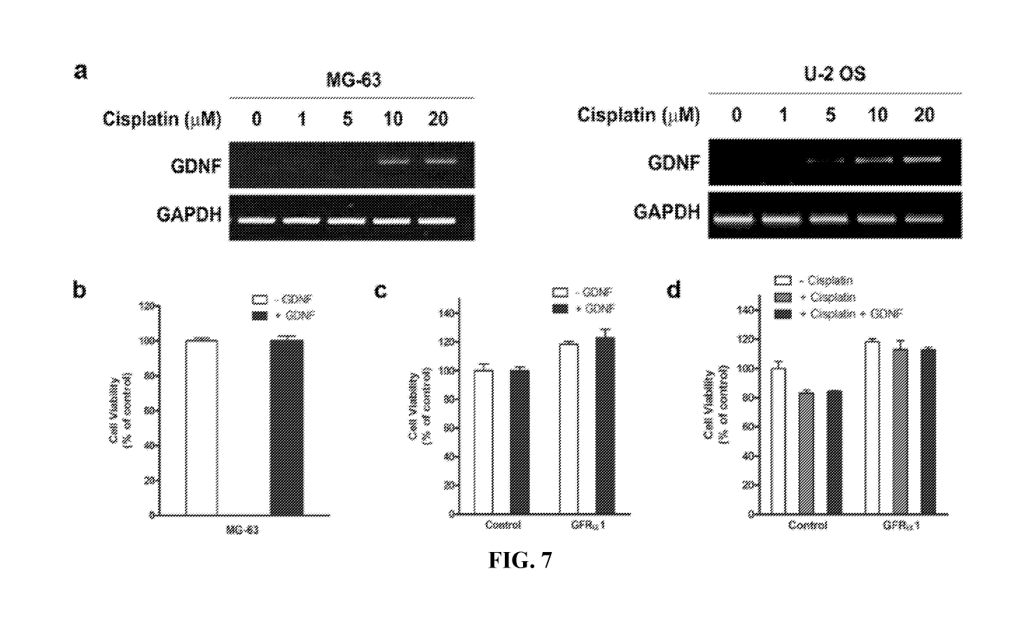

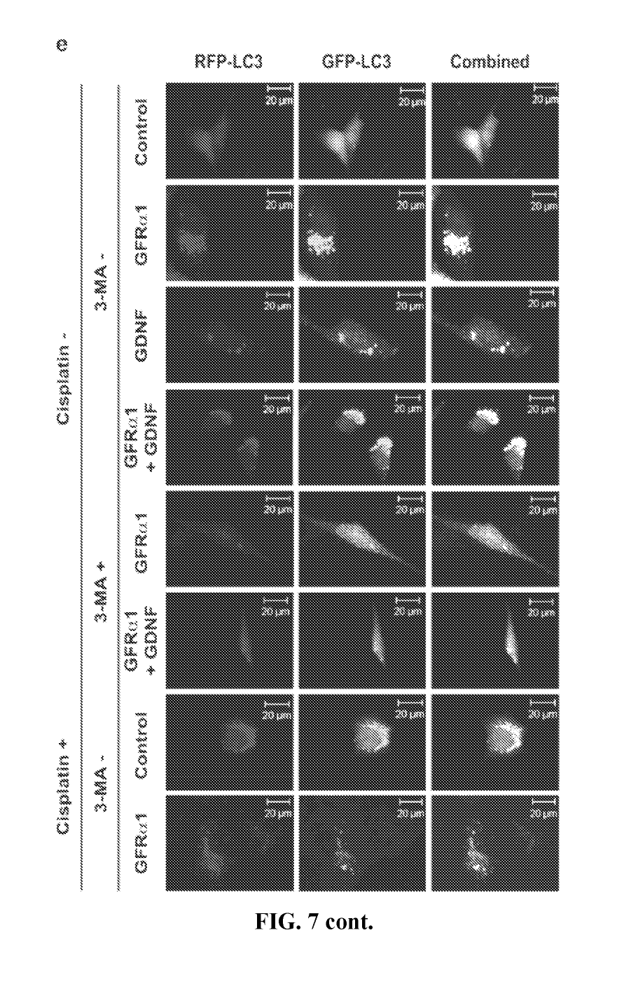

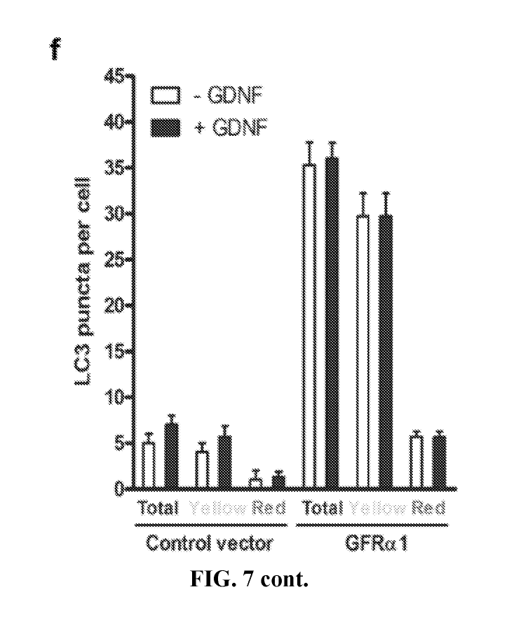

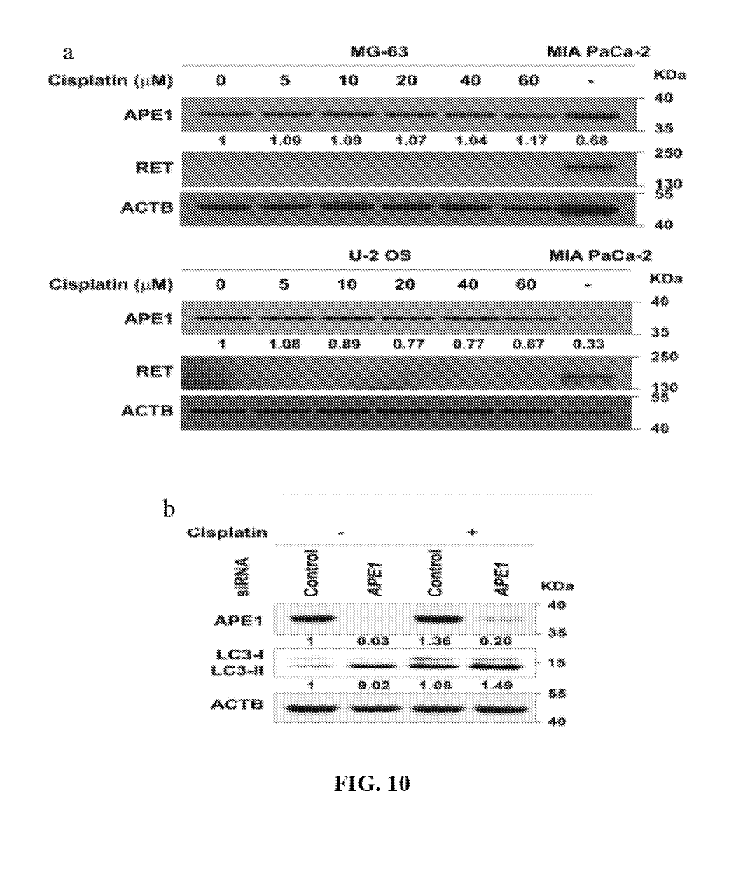

However, GDNF did not have an effect on GFRα1-mediated cell survival and autophagy after cisplatin treatment (FIGS. 7d and 7e), indicating that cisplatin-induced autophagy proceeds through GFRα1 signaling independently of GDNF. This data suggests that other ligands may be induced by cisplatin which bind GFRα1 to activate autophagy. Additionally, given that tyrosine kinases are important in cancer development, research has focused on the role of RET in GDNF signaling within cancer (51). However, in the investigation, RET expression was not detected in either osteosarcoma or cisplatin-treated osteosarcoma cells (FIG. 10a).

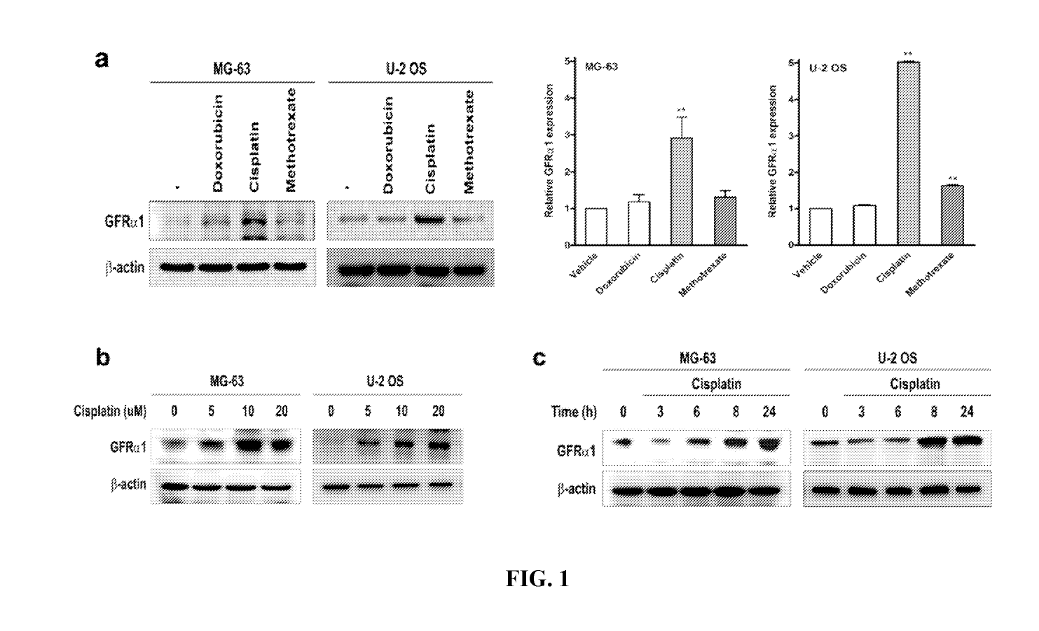

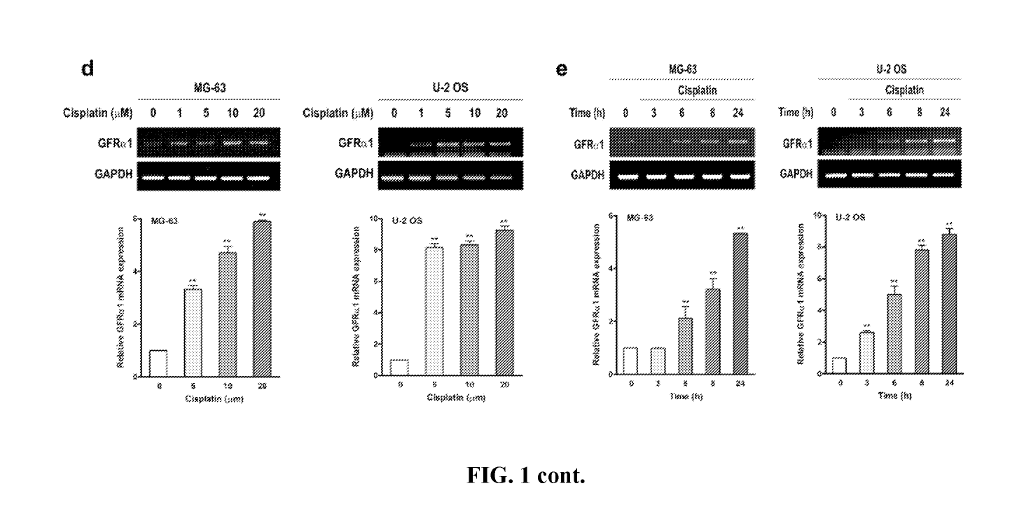

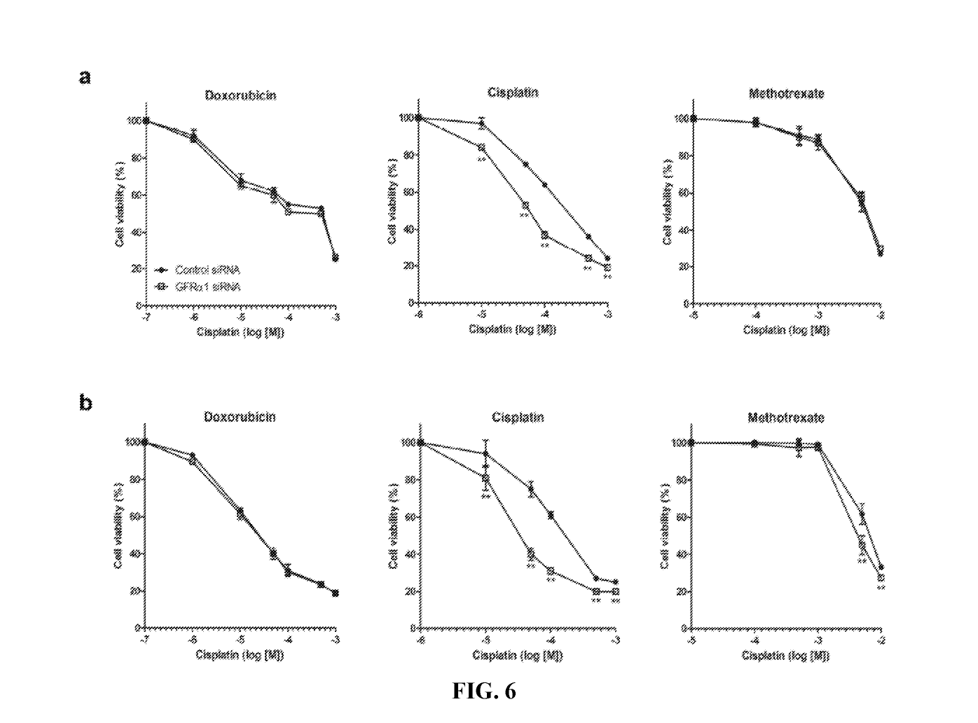

In the study, the effects of three chemotherapeutic agents (cisplatin, doxorubicin, and methotrexate) on GFRα1 expression were examined (52). Interestingly, only cisplatin increased the level of GFRα1 expression in osteosarcoma (FIG. 1a). Consistently, GFRα1 deficiency increased the sensitivity of osteosarcoma to cisplatin but not to the other two agents (FIG. 6). Previous studies showed that all three agents are able to induce HMGB1 expression and autophagy in osteosarcoma cells, which can increase chemoresistance (53); therefore the current findings suggest that all three agents can contribute to chemoresistance in osteosarcoma through different autophagy signaling mechanisms.

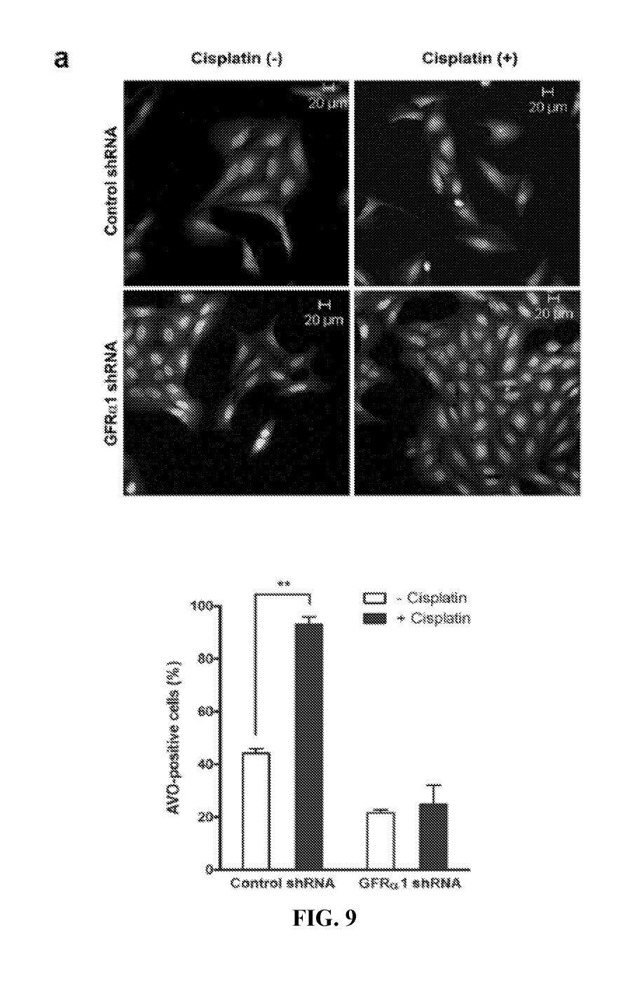

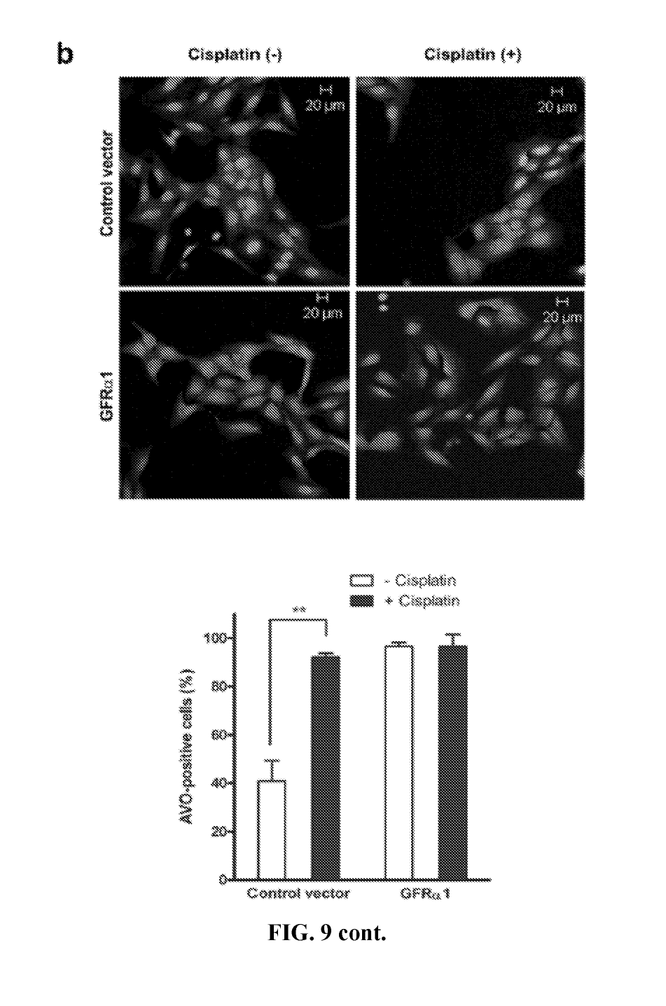

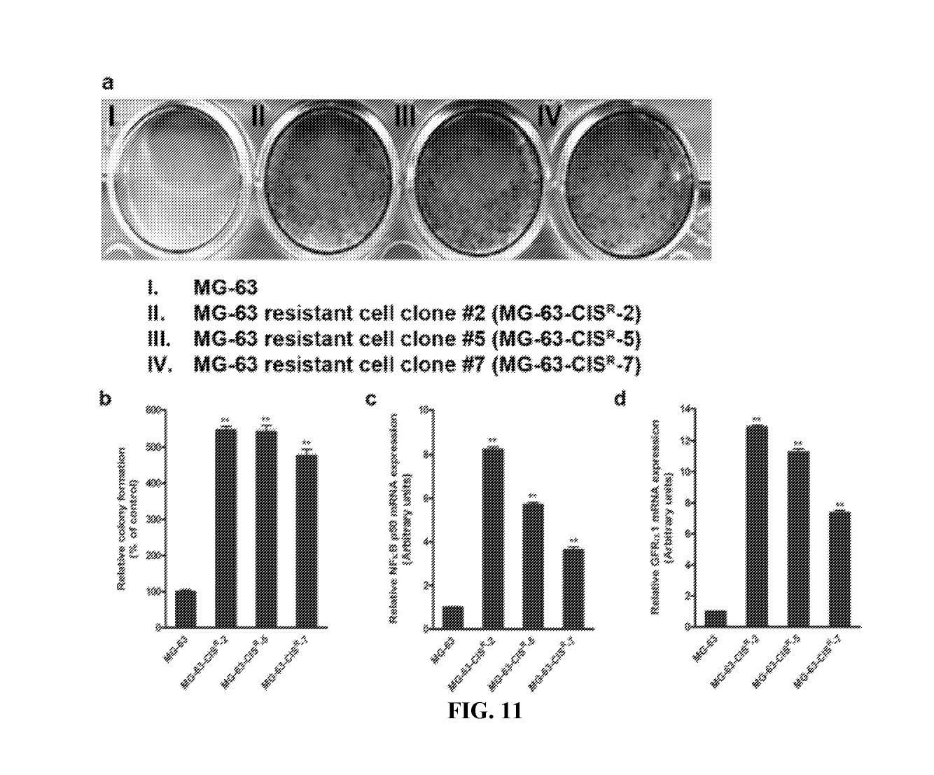

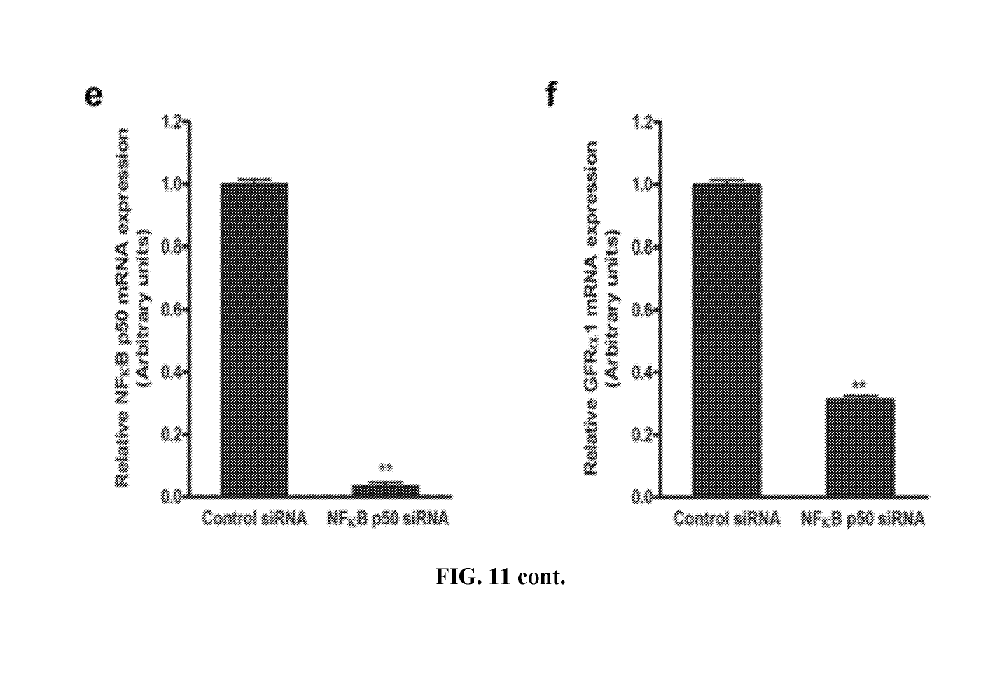

APE1 overexpression was observed in osteosarcoma patients treated with chemotherapy, suggesting its involvement in chemoresistance of osteosarcoma (54). Previous work showed that APE1 expression promoted pancreatic cancer progression by up-regulating GFRα1 expression via activation of NFκB p50 (27). The findings showed that APE1 expression was not induced in osteosarcoma cells after cisplatin treatment (FIG. 10a). Similarly, knockdown of APE1 does not affect the level of LC3-II expression or the accumulation of AVO-positive cells following cisplatin treatment (FIG. 10b and FIG. 9b). Interestingly, knockdown of APE1 in MG-63 cells increased the level of LC3-II expression and AVO-positive cells compared to control cells without treatment (FIG. 10b and FIG. 9b), suggesting APE1 has a function in autophagy unrelated to the GFRα1/SRC/AMPK mechanism induced by cisplatin. Additionally, the results revealed NFκB p50 plays a role in regulating GFRα1 expression in cisplatin-resistant MG-63 cell lines (FIG. 11a and FIG. 11b). The data suggests that cisplatin treatment induces GFRα1 expression through NFκB signaling independently of APE1.

GFRα1 is expressed in several human cancers and it is involved in tumorigenesis through the regulation of migration and invasion. Therefore, it was of interest to determine whether GFRα1 also has a role in the development of chemoresistance in other types of cancers, in addition to osteosarcoma. It is also possible that other chemotherapeutic agents can induce GFRα1 expression to trigger autophagy in cancer cells, even though doxorubicin and methotrexate did not induce its expression in osteosarcoma cells.

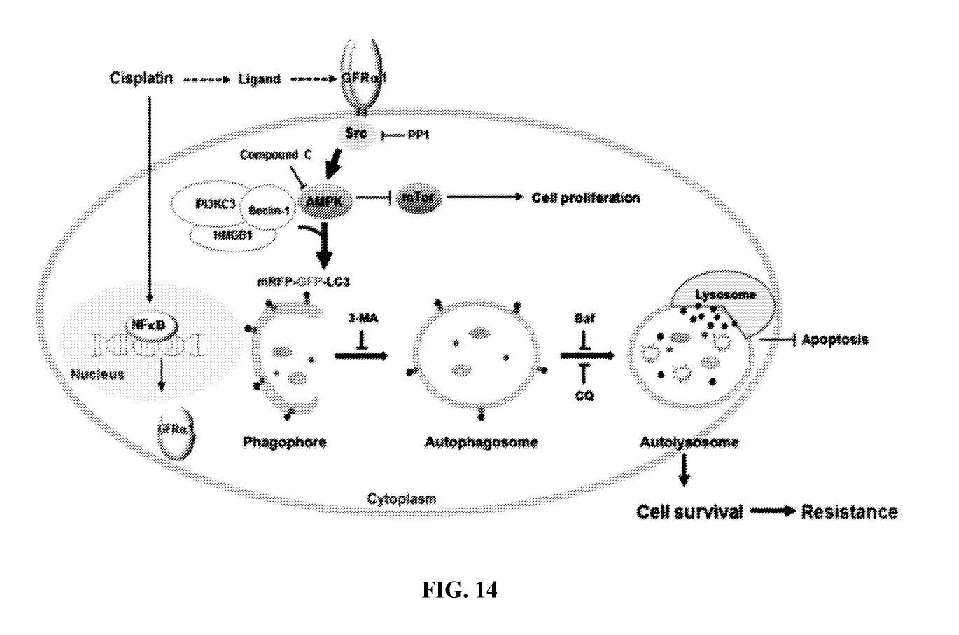

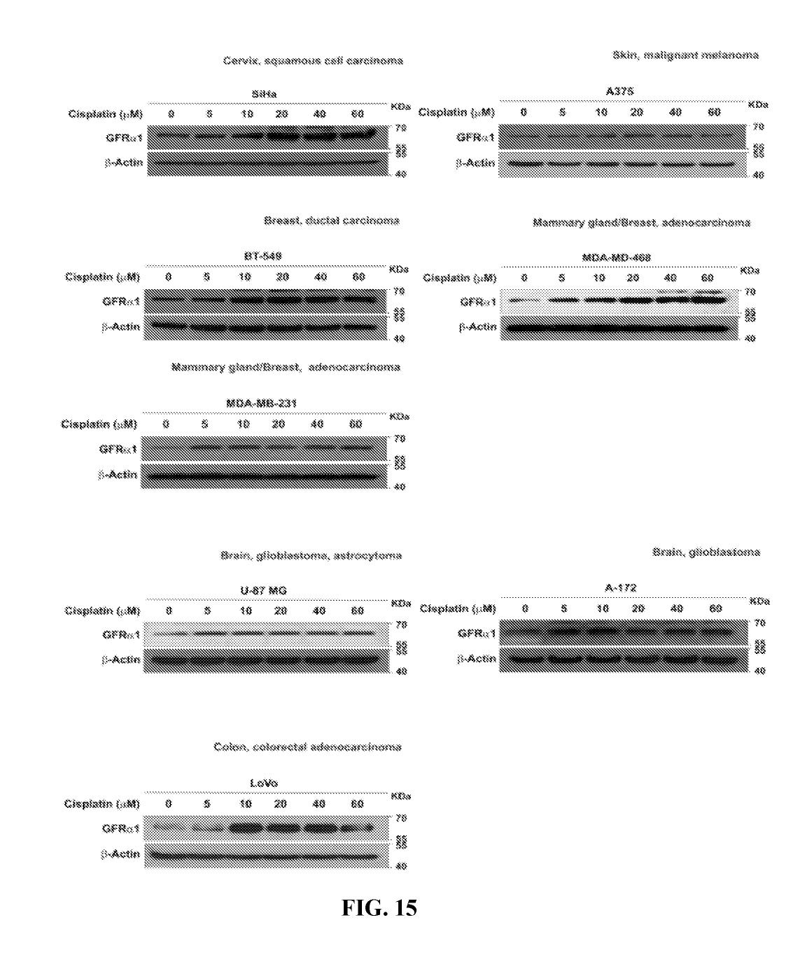

Taken together, the data showed that GFRα1 is a critical regulator in cisplatin-induced chemoresistance of osteosarcoma. Increased expression of GFRα1 by cisplatin, via NFκB signaling, induced the phosphorylation of its downstream kinase SRC and subsequently enhanced AMPK/mTOR-mediated autophagy, which contributes to chemoresistance (FIG. 14). The study described herein suggests that GFRα1 could be a potential therapeutic target for the prevention of chemoresistance in osteosarcoma and possibly other types of cancers as well.

In certain aspects of the invention GFRα1 can be used as a biomarker for cisplatin resistance. A biomarker is an organic biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. As such, they are useful as markers for disease (diagnostics), therapeutic effectiveness of a drug (theranostics) and of drug toxicity.

Aspects of the current invention seeks to develop methods for identifying patients having or at risk of developing a cancer resistant to a chemotherapy, including biochemical assays and gene expression profiling. In certain aspects, the biomarkers of this invention can be measured or detected by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies.

Aspects of this invention contemplate traditional immunoassays including, for example, sandwich immunoassays including ELISA or fluorescence-based immunoassays, as well as other enzyme immunoassays. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

In particular embodiments a sample is examined using immunohistochemistry and staining protocols. Immunohistochemical staining of tissue sections has been shown to be a reliable method of assessing or detecting presence of proteins in a sample. Immunohistochemistry (“IHC”) techniques utilize an antibody to probe and visualize cellular antigens in situ, generally by chromogenic or fluorescent methods.

For sample preparation, a tissue or cell sample from a mammal (typically a human patient) may be used. Examples of samples include, but are not limited to, cancer cells such as bone, colon, brain, breast, prostate, ovary, cervix, skin. lung, stomach, pancreas, lymphoma, and leukemia cancer cells. The sample can be obtained by a variety of procedures known in the art including, but not limited to surgical excision, aspiration or biopsy. The tissue may be fresh or frozen. In one embodiment, the sample is fixed and embedded in paraffin or the like. The tissue sample may be fixed (i.e. preserved) by conventional methodology (See e.g., The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). By way of example, neutral buffered formalin, Bouin's or paraformaldehyde, may be used to fix a sample.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used. Alternatively, commercially available deparaffinizing non-organic agents such as Hemo-De7 (CMS, Houston, Tex.) may be used.

Optionally, subsequent to the sample preparation, a tissue section may be analyzed using IHC. IHC may be performed in combination with additional techniques such as morphological staining and/or fluorescence in-situ hybridization. Two general methods of IHC are available; direct and indirect assays. According to the first assay, binding of antibody to the target is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody. The primary and/or secondary antibody used for immunohistochemistry typically will be labeled with a detectable moiety. Numerous labels are available which can be generally grouped into the following categories: (a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting. (b) Colloidal gold particles. (c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example. Fluorescence can be quantified using a fluorimeter. (d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Examples of enzyme-substrate combinations include, for example: (a) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); (b) alkaline phosphatase (AP) with para-Nitrophenyl phosphate as chromogenic substrate; and (c) β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

Numerous other enzyme-substrate combinations are available to those skilled in the art. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the sample preparation procedures discussed above, further treatment of the tissue section prior to, during or following IHC may be desired, For example, epitope retrieval methods, such as heating the tissue sample in citrate buffer may be carried out.

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above.

### I. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### GFRα1 Promotes Cisplatin-Induced Chemoresistance in Osteosarcoma by Inducing Src/AMPK-Mediated Autophagy

In this study, it is shown that glial cell line-derived neurotrophic factor (GDNF) receptor al, or GFRα1, contributes to cisplatin-induced chemoresistance in osteosarcoma. The level of GFRα1 mRNA and protein expressions were increased in human osteosarcoma cells after cisplatin treatment. Knockdown of GFRα1 expression significantly enhanced cisplatin-induced apoptotic cell death, while overexpression of GFRα1 reduced it. Additionally, GFRα1 expression significantly increased cisplatin-induced autophagy, and inhibition of autophagy in GFRα1-overexpressing cells restored the chemosensitivity to cisplatin. GFRα1 induced Src phosphorylation and then AMP-activated protein kinase (AMPK) phosphorylation, in cisplatin-treated osteosarcoma cells, resulted in increased autophagy. Inhibition of Src or AMPK activation repressed cisplatin-induced autophagy and further reduced cell viability. GFRα1 expression promoted tumor formation and growth in mouse xenograft models and injection of chloroquine, an autophagy inhibitor, in GFRα1-overexpressing tumors inhibited autophagy and significantly reduced tumor growth. Moreover, treatment period and metastatic status in human patients treated with cisplatin is associated with increased expression of GFRα1. These findings suggest that autophagy by GFRα1/Src/AMPK axis is a promising target for novel therapeutic approaches in overcoming cisplatin-induced chemoresistance in osteosarcoma.

A. Results

GFRα1 expression is induced by cisplatin in osteosarcoma cells. Aberrant expression of GFRα1 has been observed in malignant human cancers and it has a role in regulating tumor cell migration and invasion, indicating that it is involved in cancer progression and metastasis (25-27). To investigate whether GFRα1 has a role in chemoresistance of osteosarcoma, the effects of doxorubicin, cisplatin, and methotrexate on GFRα1 expression were examined in two well-known osteosarcoma cell lines, MG-63 and U-2 OS. Cisplatin significantly increased the level of GFRα1 expression in both cell lines, whereas doxorubicin and methotrexate showed no or little effect on its expression (FIG. 1a). The effect of cisplatin on GFRα1 expression was dose and time dependent (FIGS. 1b and 1c). Cisplatin also increased the level of GFRα1 mRNA in both cell lines in a dose and time dependent manner (FIGS. 1d and 1e), indicating that GFRα1 expression is induced by cisplatin in osteosarcoma cells at both transcriptional and translational levels. To examine the effects of GFRα1 expression on the efficacy of the chemotherapeutic agents, GFRα1 was knockdowned by GFRα1-specific small interfering RNA (siRNA) in both MG-63 and U-2 OS cells and then the cell lines were treated with each of the three agents. Cisplatin treatment significantly reduced cell proliferation in both GFRα1-deficient MG-63 and U-2 OS cells compared to control cells, whereas doxorubicin and methotrexate showed little or no effect on cell proliferation of either GFRα1-deficient cell lines (FIGS. 6a and 6b). Given that GDNF is the major ligand of GFRα1, the effect of cisplatin on GDNF expression was also examined. An increase in GDNF mRNA expression was detected after treatment with 10 and 20 μM of cisplatin treatment in osteosarcoma cells (FIG. 7a). However, GDNF had no effect on cell proliferation of MG-63 cells (FIG. 7b). The results imply GFRα1 inhibits cisplatin-induced apoptosis and this effect is independent of the GFRα1 ligand GDNF.

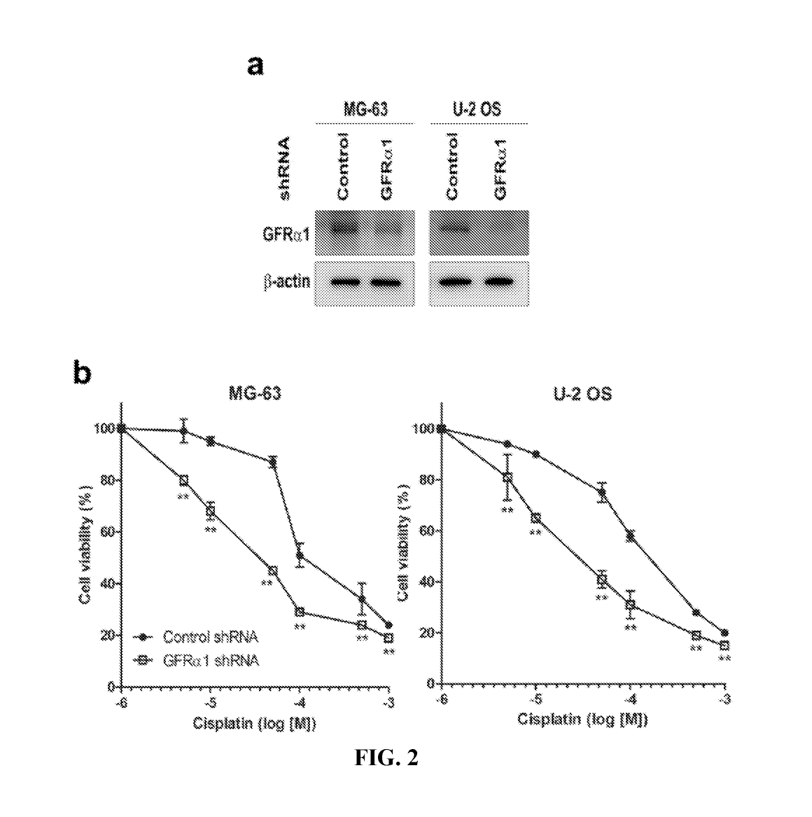

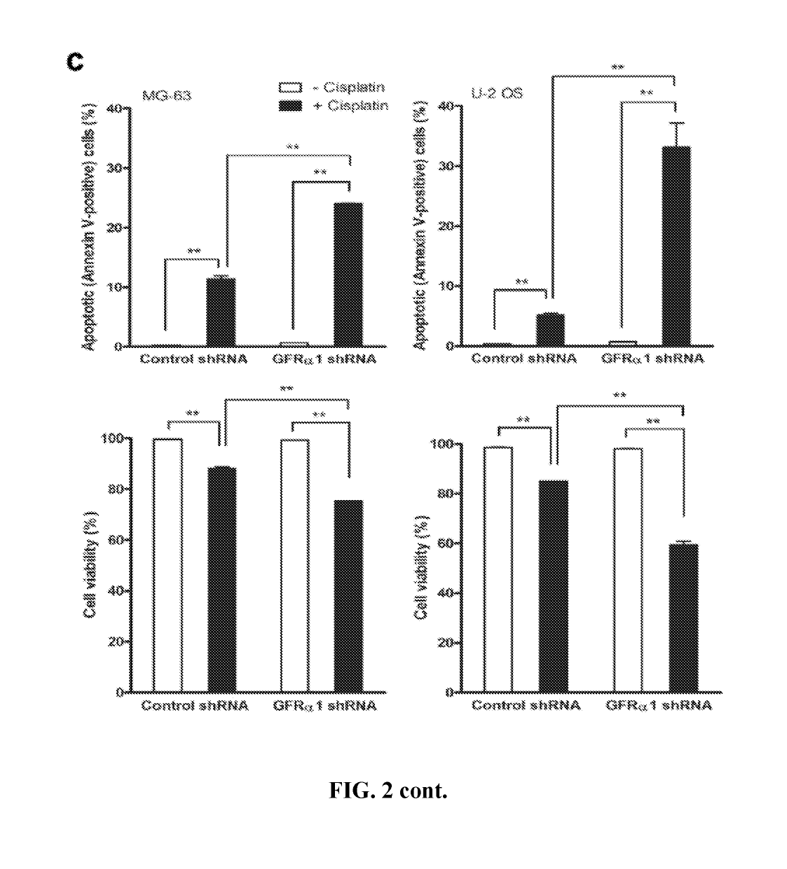

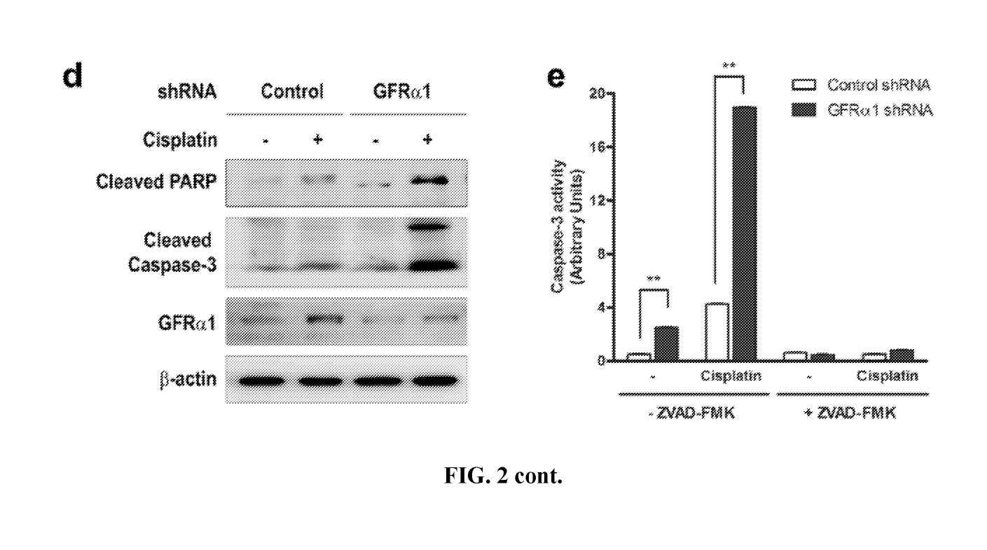

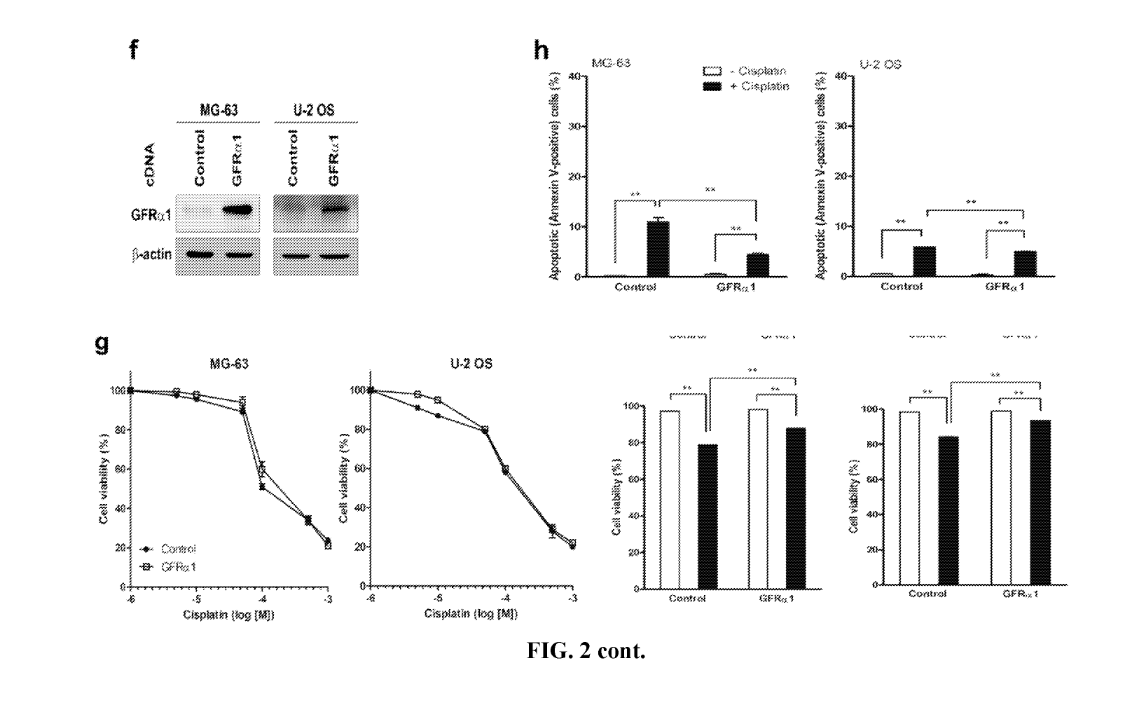

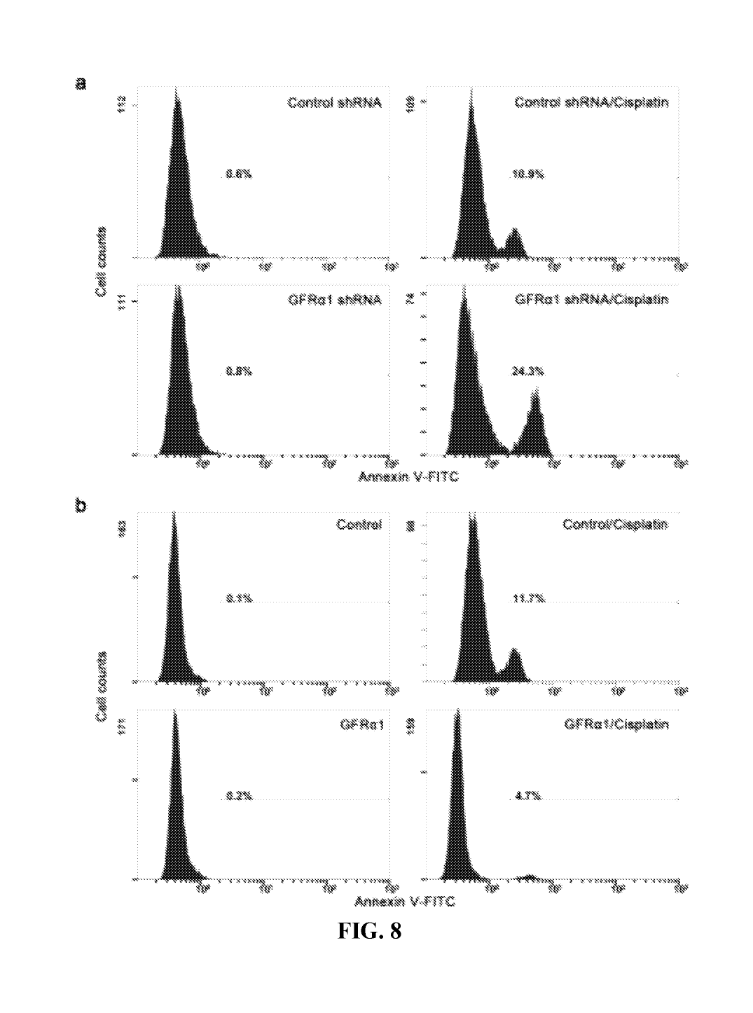

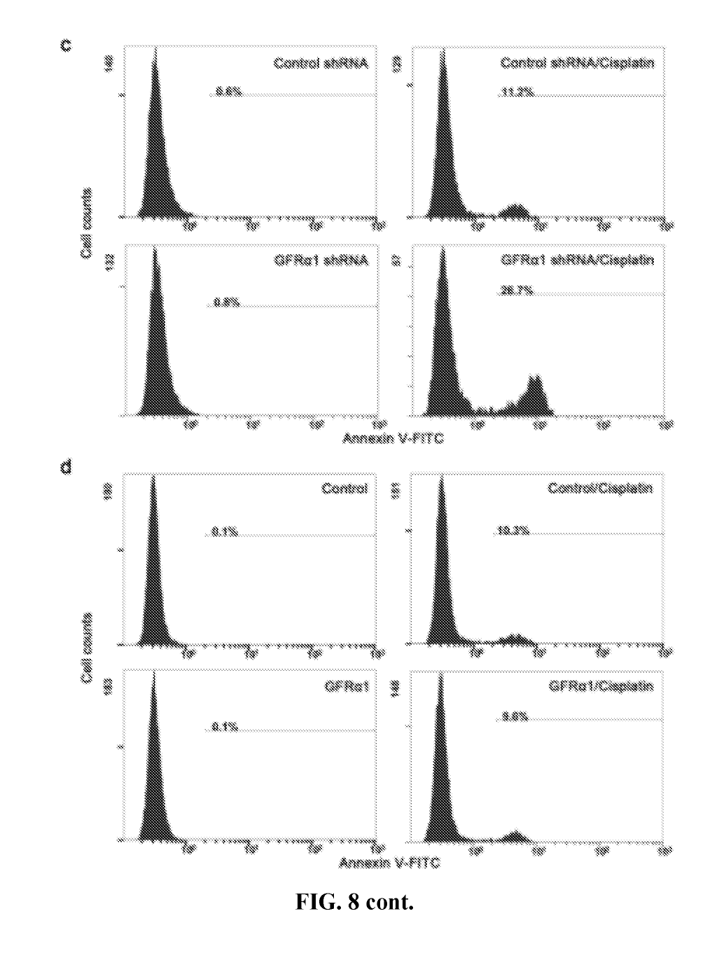

GFRα1 expression reduces efficacy of cisplatin in osteosarcoma cells. To further investigate the role of GFRα1 in cisplatin-induced apoptosis, stable GFRα1-deficient MG-63 and U-2 OS cell lines were generated using GFRα1-specific small hairpin RNA (shRNA). Knockdown of GFRα1 expression led to a decrease of GFRα1 protein levels in both osteosarcoma cell lines (FIG. 2a). Like siRNA-mediated knockdown of GFRα1, stable knockdown of GFRα1 significantly reduced cell proliferation of cisplatin-treated osteosarcoma cells in a dose-dependent manner compared to control cells (FIG. 2b). Furthermore, knockdown of GFRα1 significantly increased cisplatin-induced apoptosis which corresponded with a significant reduction in cell viability, indicating GFRα1-deficient cells are more sensitive to cisplatin treatment (FIG. 2c and FIG. 8a). Western blot analysis with the apoptotic markers cleaved PARP and cleaved caspase-3 confirmed this result as evidenced by a significant increase in both cleaved PARP and cleaved caspase-3 expression levels in GFRα1-deficient MG-63 cells after cisplatin treatment (FIG. 2d). Consistently, caspase-3 activity significantly increased in GFRα1-deficient MG-63 cells, and it increased even more dramatically upon treatment with cisplatin in GFRα1-deficient MG-63 cells compared to cisplatin treated control cells (FIG. 2e). Addition of the pan caspase inhibitor ZVAD-FMK reversed this effect (FIG. 2e), indicating loss of GFRα1 stimulates apoptosis, particularly in the presence of cisplatin. Additionally, stable MG-63 and U-2 OS cell lines that overexpress human GFRα1 were generated by transfection with a human GFRα1 expression vector (FIG. 2f). Overexpression of GFRα1 did not lead to an increase in cell proliferation of either cisplatin-treated osteosarcoma cell line compared to control in dose response experiments (FIG. 2g). However, further analysis using FACS showed that overexpression of GFRα1 significantly reduced cisplatin-induced apoptosis which corresponded with an increase in cell viability in both cell lines (FIG. 2h and FIG. 8b). Together, these results demonstrate that GFRα1 reduces the susceptibility of osteosarcoma cells to cisplatin-induced apoptosis.

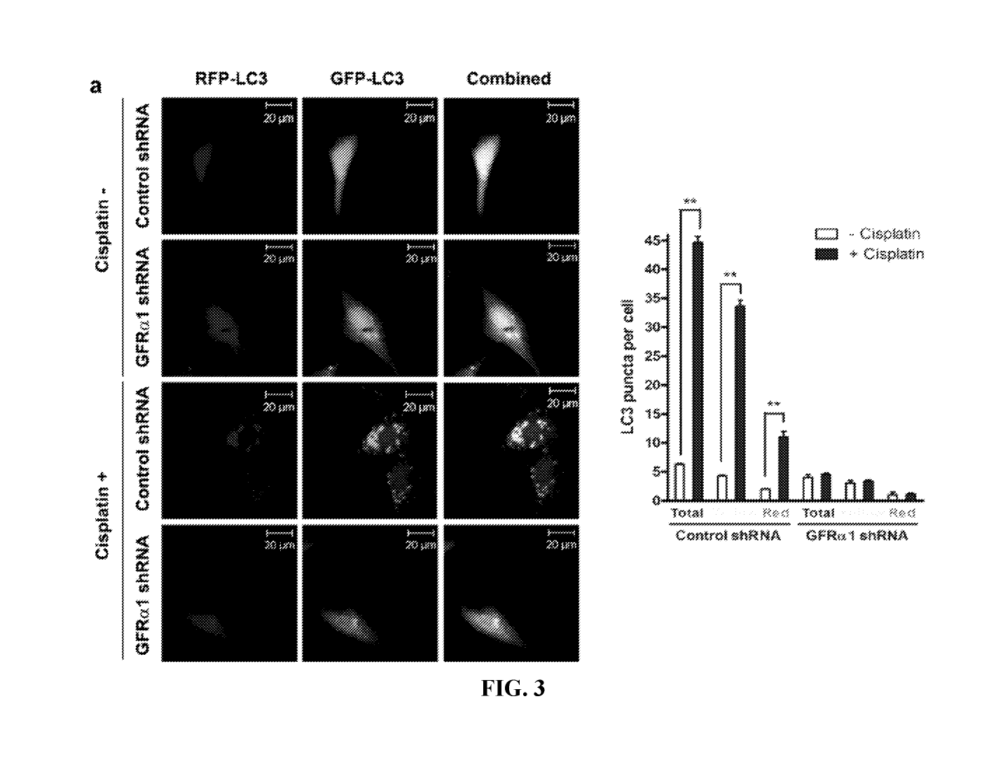

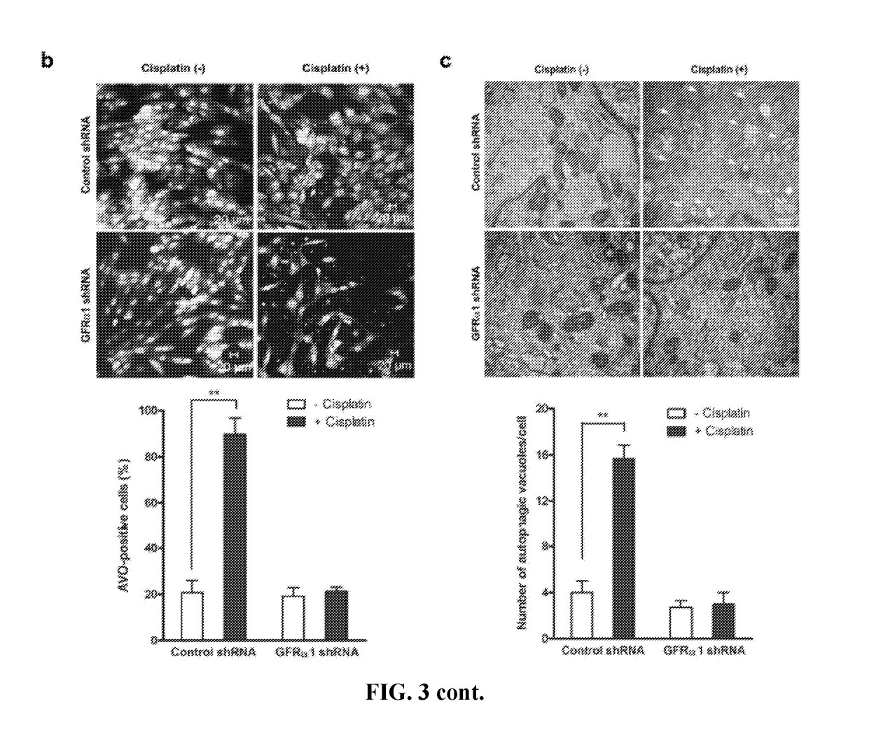

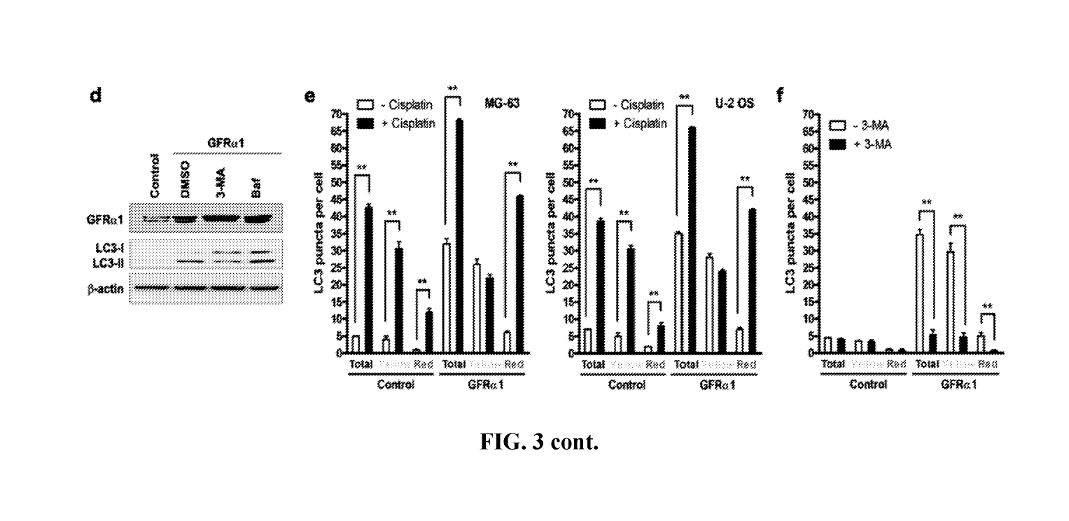

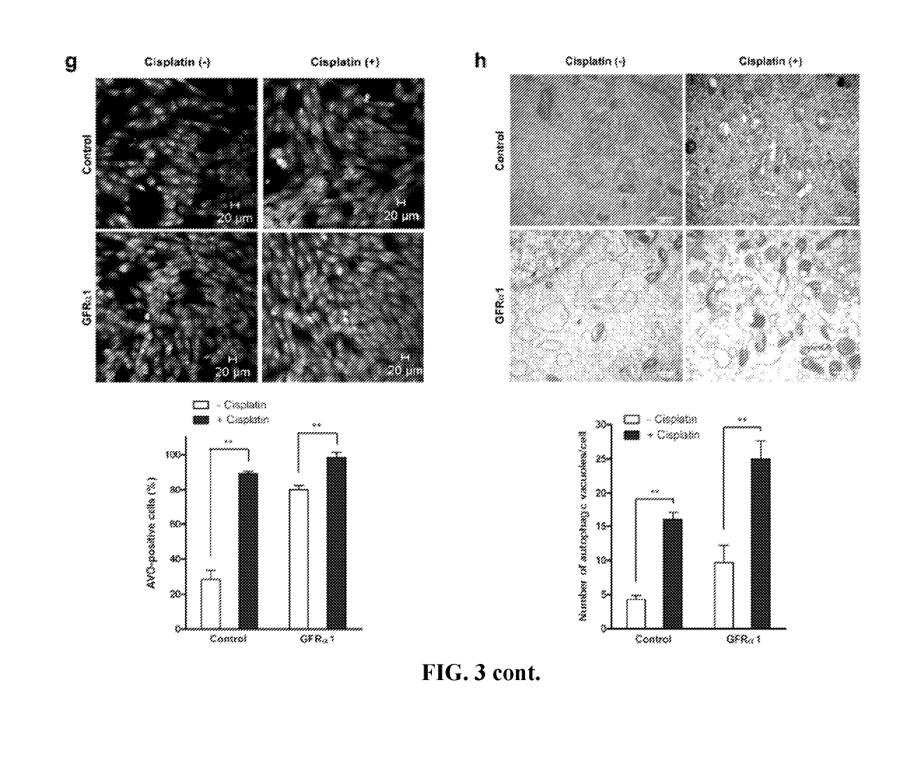

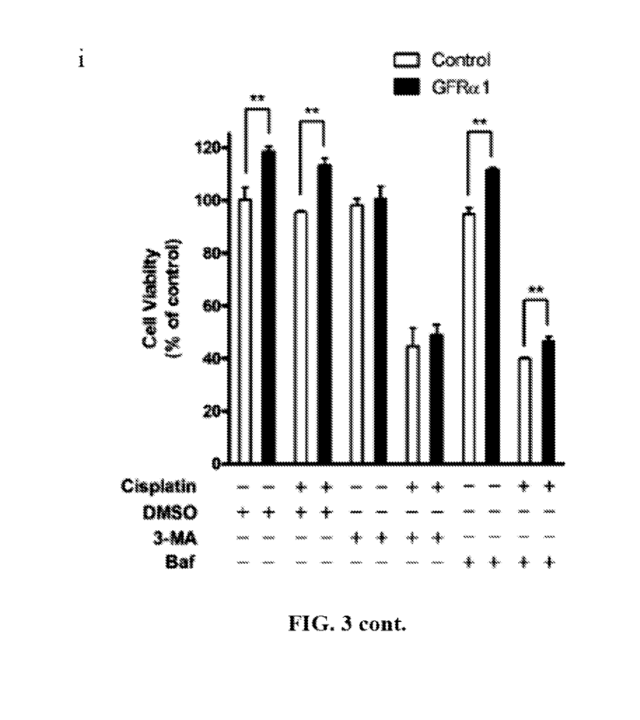

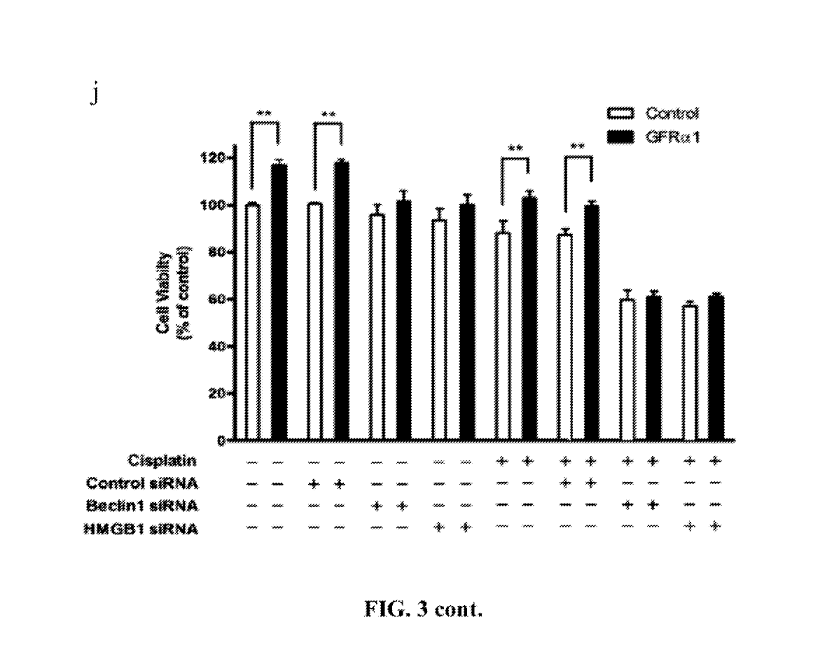

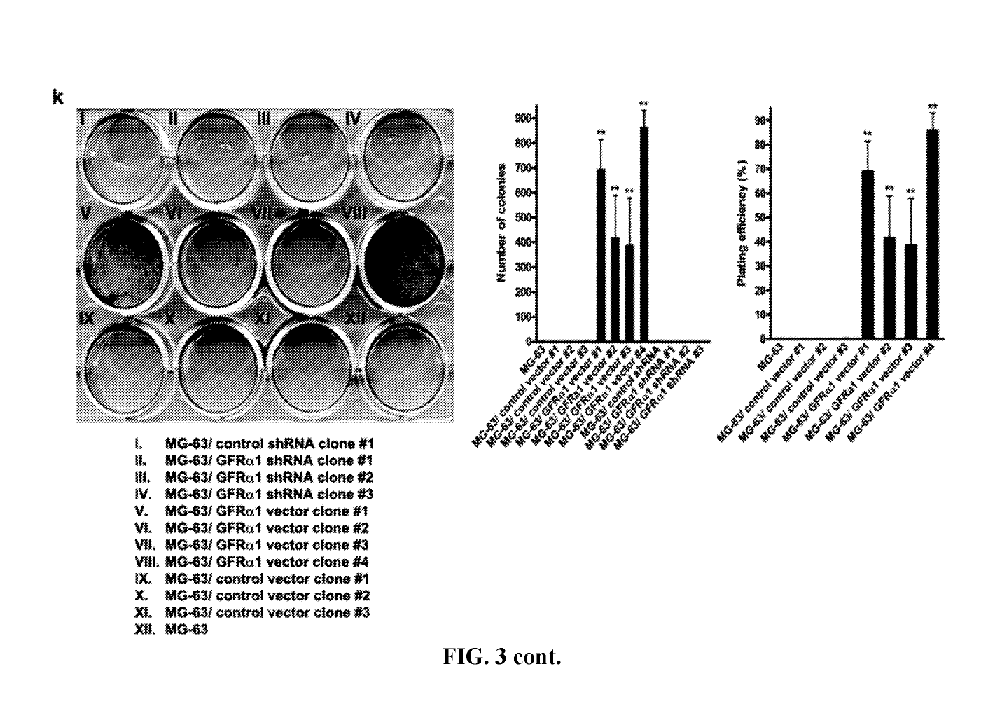

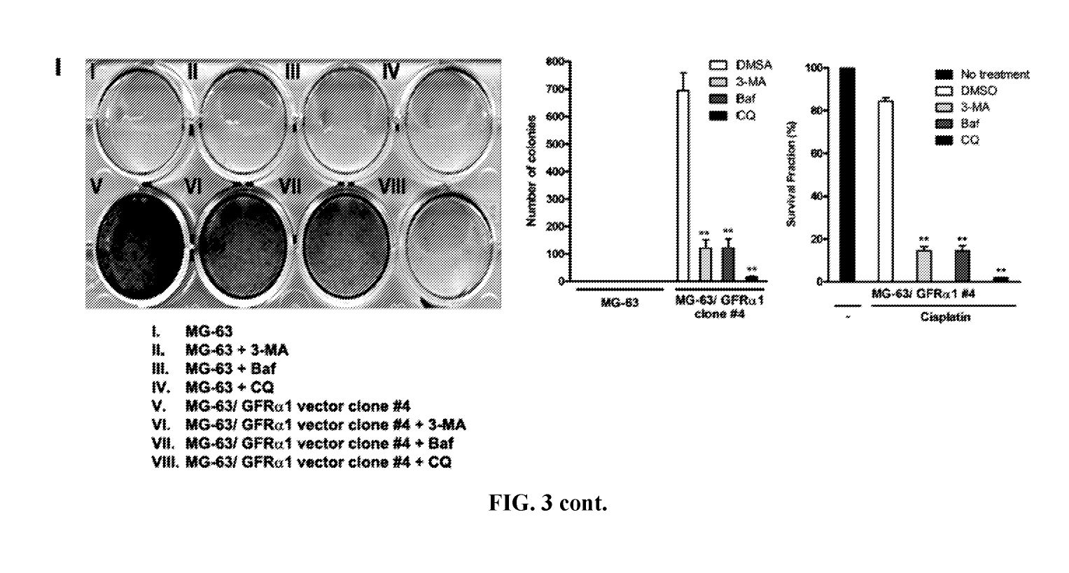

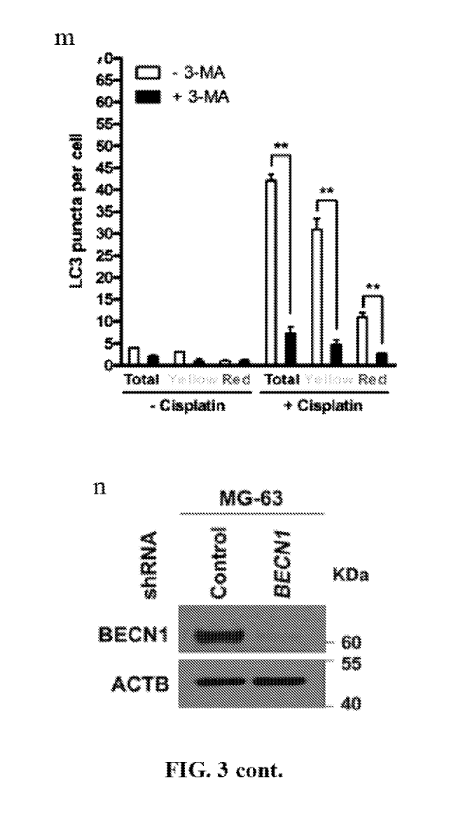

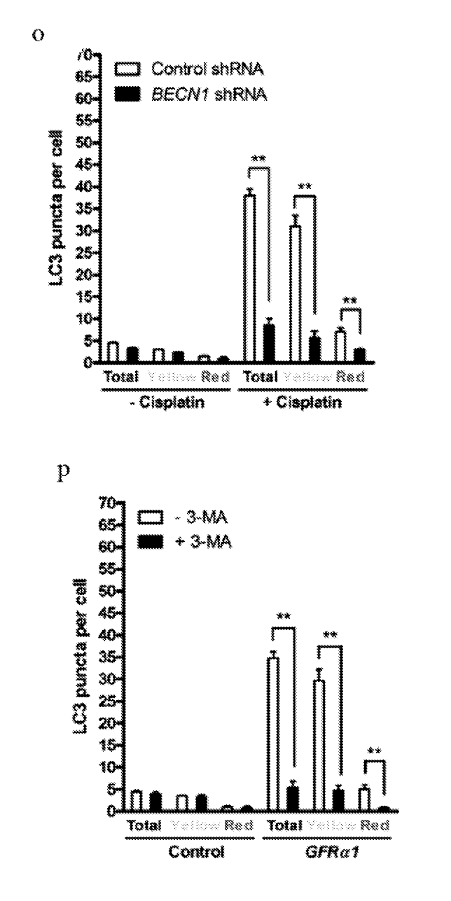

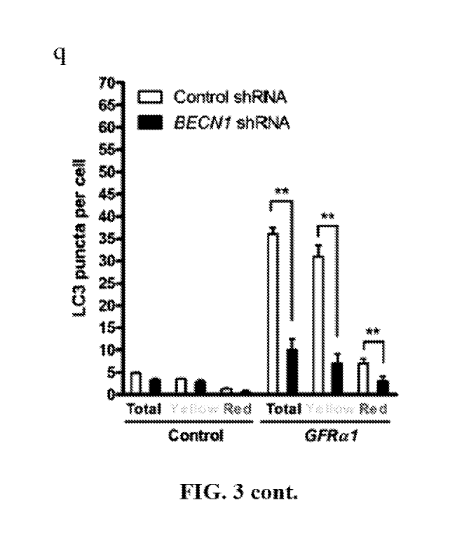

GFRα1 triggers autophagy in response to cisplatin. Autophagy is a mechanism that can promote resistance to apoptosis and potentially chemotherapy which works by triggering cell death. Therefore, what role autophagy might play in cisplatin-induced apoptosis of osteosarcoma cells in relation to GFRα1 expression was assessed. First investigated was whether GFRα1 deficiency regulates microtubule-associated protein light chain 3 (LC3) puncta formation, which exists on autophagosomes and is widely used as a marker for autophagy (28). Fluorescent imaging analysis of LC3 puncta formation using a mRFP-GFP-LC3 reporter (a RFP-GFP tandem fluorescent-tagged LC3 vector) showed that cisplatin treatment significantly increased LC3 puncta formation in MG-63 cells transfected with control shRNA, while there was no change in puncta formation in GFRα1-deficient cells (FIG. 3a). In particular, cisplatin treatment increased both RFP/GFP-positive yellow puncta (autophagosomes) and RFP-positive red puncta (autolysosomes) formation in control cells, and the number of RFP/GFP-positive yellow puncta was greater than the number of RFP-positive red puncta (FIG. 3a). Acridine orange staining of GFRα1-deficient MG-63 cells showed that knockdown of GFRα1 did not increase the accumulation of acidic vesicular organelle (AVO)-positive cells following cisplatin treatment, while treatment of control cells with cisplatin significantly increased AVO-positives cells (FIG. 3b). This result was also observed in GFRα1-deficient U-2 OS cells (FIG. 9a). Moreover, ultrastructural analysis by transmission electron microscopy (TEM) revealed that the number of autophagic vacuoles per cell was markedly increased in control cells following cisplatin treatment, whereas GFRα1 deficiency had no effect on autophagic vacuole formation in cells after similar treatment (FIG. 3c). Addition of 3-methyladenine (3-MA), an inhibitor of early-phase autophagy, blocked cisplatin-induced puncta formation in MG-63 cells (FIG. 3m). To further confirm cisplatin-induced autophagy, stable BECN1/Beclin 1-deficient MG-63 cell line was generated using BECN1-specific shRNA. Knockdown of BECN1 expression led to a decrease of BECN1 protein levels in MG-63 cells (FIG. 3n). Similar to 3-MA treatment, stable knockdown of BECN1 significantly reduced puncta formation after cisplatin treatment compared to cisplatin treated control cells (FIG. 3o). The data suggests that cisplatin induces autophagy in osteosarcoma and GFRα1 is required for this autophagic response.

To further investigate the involvement of GFRα1 in cisplatin-induced autophagy and its effect on the development of cisplatin resistance, we examined the conversion of LC3-I to LC3-II which occurs during autophagosome formation (9,11,12,29). Western blot analysis showed an increase in the level of LC3-II protein expression in GFRα1-overexpressing MG-63 cells in comparison to control (FIG. 3d), indicating that overexpression of GFRα1 triggers autophagy. Addition of 3-methyladenine (3-MA), an inhibitor of early-phase autophagy, reduced the level of LC3-II expression, whereas bafilomycin A1 (Baf), an inhibitor of late-phase autophagy, increased LC3-II expression levels (FIG. 3d). Consistent with these results, LC3 puncta formation following cisplatin treatment was significantly increased in both GFRα1-overexpressing MG-63 and U-2 OS cells compared to their control cells (FIG. 3e and FIG. 7e). Whereas the number of RFP/GFP-positive yellow puncta was greater than the number of RFP-positive red puncta in cisplatin-treated control cells, the number of RFP-positive red puncta was much greater than the number of RFP/GFP-positive yellow puncta in GFRα1-overexpressing cells (FIG. 3e and FIG. 7e), suggesting that GFRα1 significantly increases autophagic flux. It is important to note that basal LC3 puncta formation without cisplatin treatment was also higher in GFRα1-overexpressing cells compared to control cells (FIG. 3e and FIG. 7e). Treatment with 3-MA blocked puncta formation in both control and GFRα1-overexpressing cells (FIG. 3f and FIG. 7e). Addition of GDNF had no significant effect in either control or GFRα1-overexpressing cells (FIGS. 7e and 7f). Similar with this observation, GFRα1 overexpression in osteosarcoma cells increased AVO-positive cells compared to control cells with or without cisplatin treatment (FIG. 3g and FIG. 9a). The number of autophagic vacuoles per cell was also significantly increased in GFRα1-overexpressing cells compared to control cells with or without cisplatin treatment (FIG. 3h).

Previous studies showed that APE1 (apurine and apyrimidine endonuclese 1) increases GDNF responsiveness by upregulating GFRα1 expression in pancreatic cancer cells (27), the inventors examined whether APE1 is involved in GFRα1-mediated autophagy after cisplatin treatment in osteosarcoma cells. Western blot analysis showed that the level of APE1 expression was not affected by cisplatin treatment (FIG. 10a). Moreover, knockdown of APE1 did not reduce LC3 puncta formation after cisplatin treatment (FIG. 10b), indicating that APE1 is not involved in this mechanism. It was also examined whether RET, the downstream kinase activated by of the GDNF/GFRα1 complex, is involved in GFRα1-mediated autophagy after cisplatin treatment in osteosarcoma cells and western blot analysis showed that RET expression was not detected in MG-63 osteosarcoma cells (FIG. 10a).

Cell proliferation in GFRα1 overexpressing cells was significantly higher in the presence or absence of cisplatin compared to control cells which correlates with the increase of autophagy observed in GFRα1 overexpressing cells and this effect was blocked by addition of 3-MA but not by Baf (FIG. 3i). The increase in cell proliferation observed in GFRα1 overexpressing cells was also blocked by silencing the autophagy proteins Beclin 1 or HMGB1 with siRNA (FIG. 3j), supporting that GFRα1-mediated autophagy following cisplatin treatment contributes to increased cell proliferation. To further demonstrate the role of GFRα1 in cisplatin-mediated autophagy and cell proliferation, control cell lines, GFRα1-deficient stable cell lines or GFRα1-overexpressing stable cell lines were cultured in the presence of cisplatin and the resultant colonies were counted. All four cell lines overexpressing GFRα1 developed a significant number of colonies after seven days of incubation and showed high plating efficiency, whereas none of the control cell lines nor the GFRα1-deficient cell lines developed colonies (FIG. 3k). Treatment of GFRα1-overexpressing MG-63 clone #4, which appeared to be highly resistant to cisplatin, with 3-MA, Baf, or chloroquine (CQ) revealed that inhibition of autophagy effectively reduced the number of colonies and its viability formation mediated by GFRα1 (FIG. 31). Three cisplatin-resistant MG-63 clones were generated by treating MG-63 cells with cisplatin for 10 days. All three cisplatin-resistant cell lines developed much higher numbers of colonies compared to control MG-63 cells (FIGS. 11a and 11b). Furthermore, the level of GFRα1 mRNA expression was significantly increased in these cell lines compared to control (FIG. 11c).

The transcription factor NFκB p50 is able to bind the GFRα1 gene promoter and thereby upregulate GFRα1 mRNA expression (27). Consistent with this data, the level of NFκB p50 mRNA expression was also significantly increased in the cisplatin-resistant cell lines compared to control cells (FIG. 11d). Knockdown of NFκB p50 mRNA expression with siRNA reduced the level of GFRα1 mRNA expression in MG-63 cells (FIGS. 11e and 11f), implying cisplatin may activate NFκB p50 in order to stimulate expression of GFRα1. Additional studies using NIH3T3 cells revealed that GFRα1 has a transforming capability. Overexpression of GFRα1 in NIH3T3 cells induced cellular transformation, as shown by focus formation. NIH3T3 cells transfected with a control empty vector did not demonstrate induced transformation (FIG. 12). Collectively, these findings support a critical role for GFRα1 in the regulation of autophagy induced by cisplatin which promotes osteosarcoma cell survival and chemoresistance.

GFRα1 regulates autophagy through Src/AMPK signaling. Src is activated by GFRα1/RET signaling (20). Studies have demonstrated that Src is activated by GFRα1 in RET-deficient cells, indicating GFRα1 can activate Src signaling in a RET-independent manner (20,30). In addition to its oncogenic functions in a variety of cancers, Src is also involved in autophagy, although its exact function in autophagy is unclear, especially in regards to the acquisition of chemoresistance (31,32). Src mediated AMPK activation is involved in the regulation of autophagy (33,34). Moreover, AMPK-mediated autophagy has been shown to be involved in cisplatin-induced chemoresistence (35).

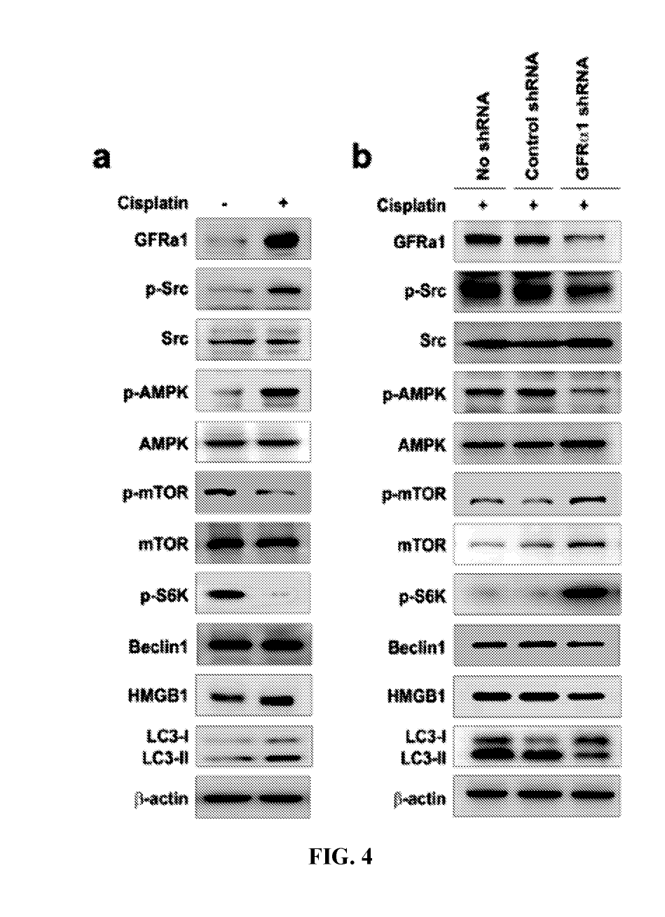

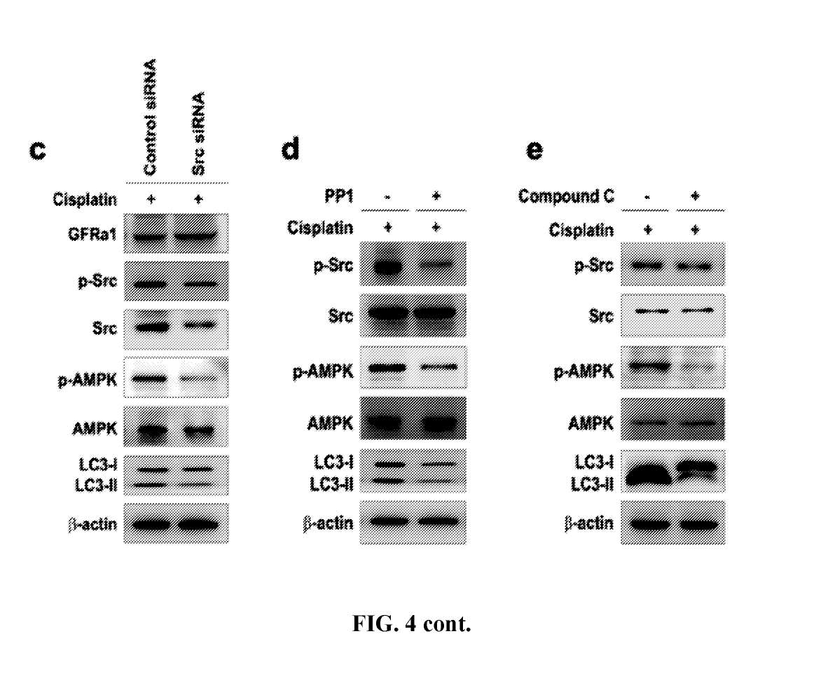

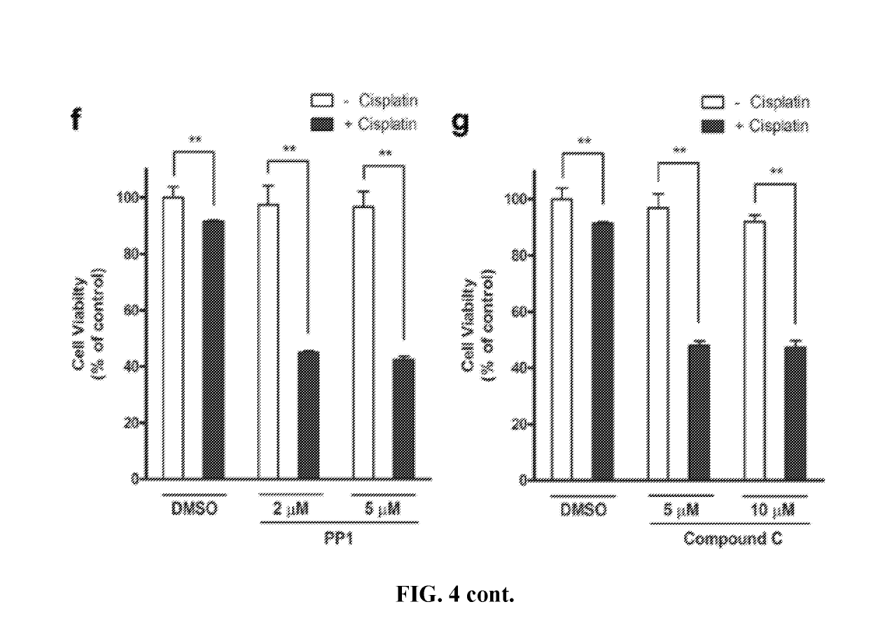

Following cisplatin treatment, the levels of phosphorylated Src and phosphorylated AMPK were increased with cisplatin-induced GFRα1 expression in MG-63 cells, and the levels of phosphorylated mTOR (mammalian target of rapamycin) and phosphorylated S6K (P70-S6Kinase), downstream kinases involved in AMPK signaling, were then decreased following AMPK activation (FIG. 4a). Up-regulation of Src/AMPK signaling by GFRα1 subsequently increased the expressions of Beclin 1, HMGB1, and LC3-II (FIG. 4a). In contrast the levels of phosphorylated Src and phosphorylated AMPK were decreased and the levels of phosphorylated mTOR and phosphorylated S6K were increased in GFRα1-deficient cells compared to control cells. Expression levels of autophagy-related proteins were then reduced in GFRα1-deficient cells compared to control cells (FIG. 4b). Inhibition of Src phosphorylation by either siRNA or its selective inhibitor PP1 led to decreased AMPK phosphoryation and LC3-II expression (FIGS. 4c and 4d). Inhibition of AMPK phosphorylation by its selective inhibitor compound C also led to decreased LC3-II expression with no effect on Src phosphorylation (FIG. 4e), confirming that AMPK is a downstream kinase of Src. Inhibition of either Src or AMPK activation by their selective inhibitors in MG-63 cells significantly reduced cell viability after cisplatin treatment compared to controls (FIGS. 4f and 4g). The data implies that following cisplatin treatment, induction of GFRα1 triggers Src activation which in turn activates AMPK signaling, leading to initiation of autophagy in osteosarcoma.

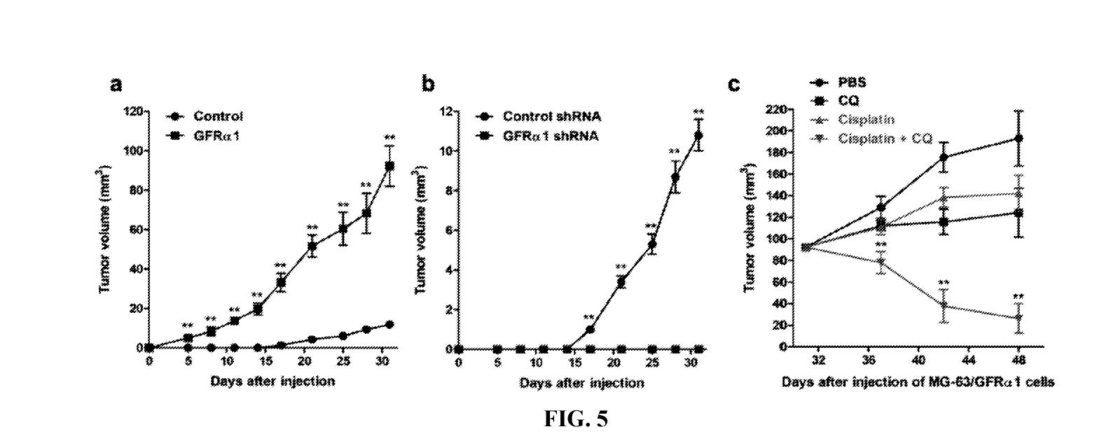

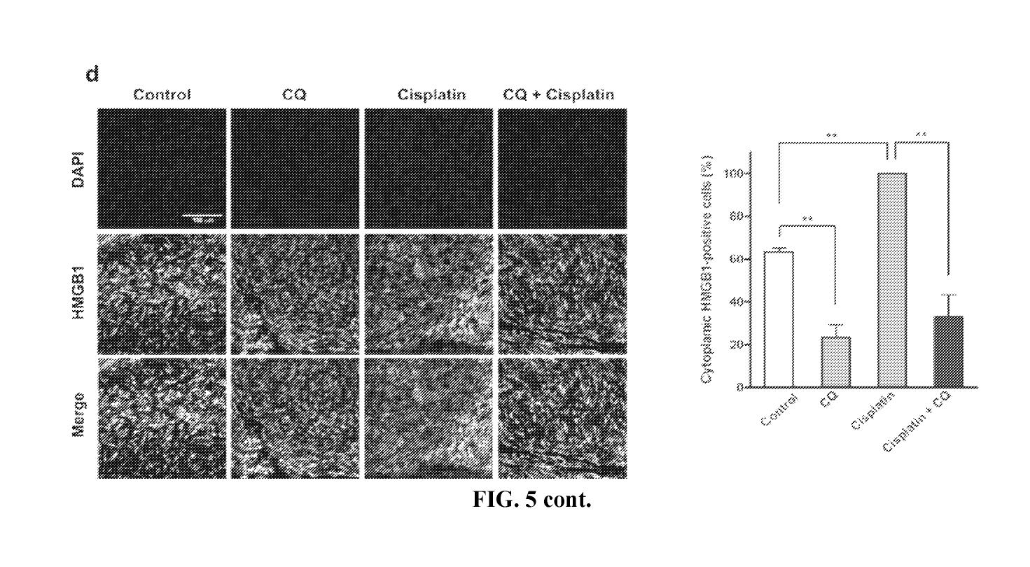

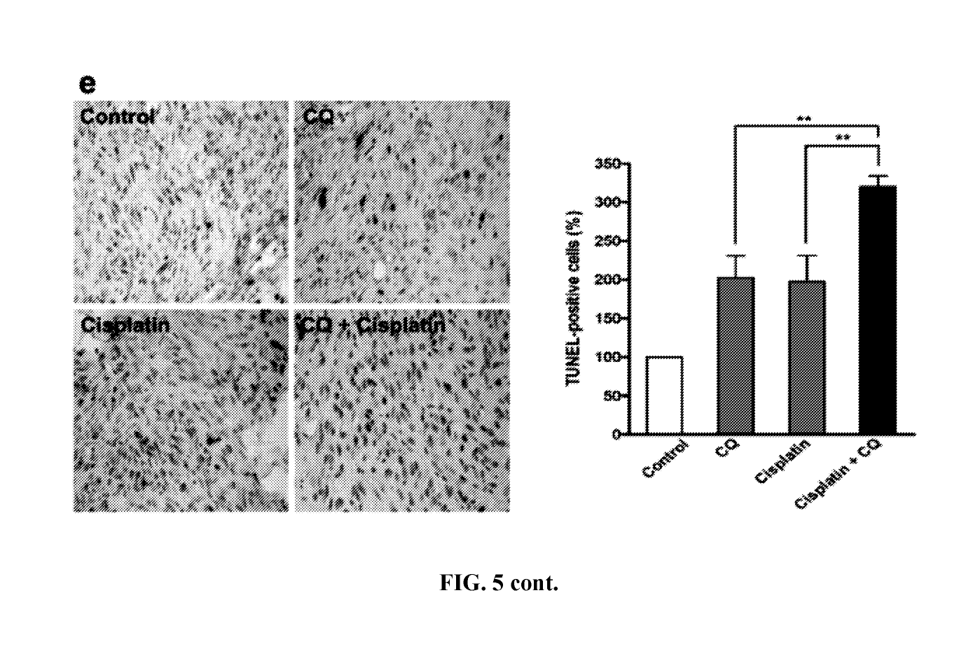

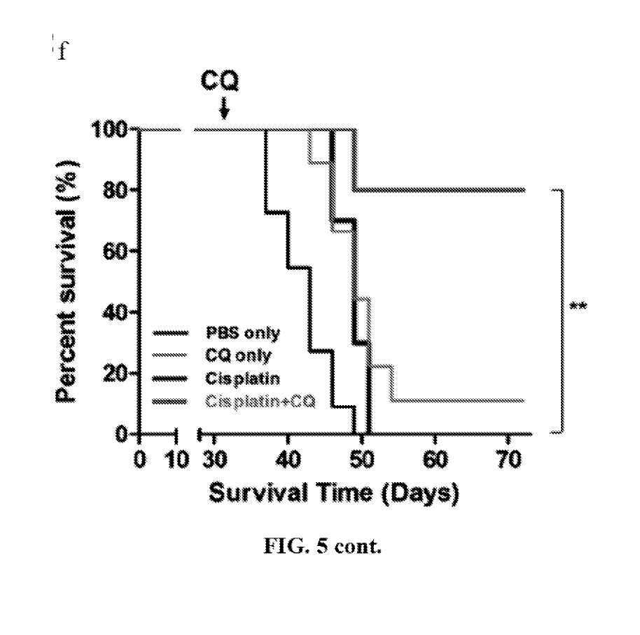

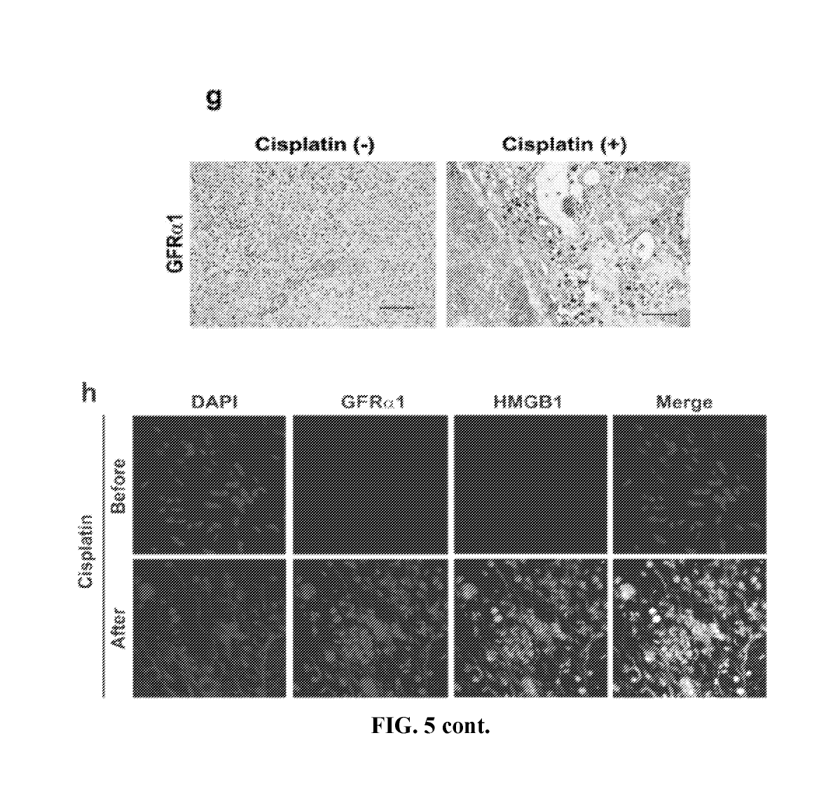

GFRα1-induced autophagy promotes tumor growth in vivo. Next the effects of GFRα1 on tumor formation were evaluated in vivo using mouse xenograft models. First, MG-63 cells or GFRα1-overexpressing MG-63cells (MG-63/GFRα1) were implanted subcutaneously in the right flank of nude mice. At five days after injection, mice injected with MG-63/GFRα1 cells started to develop tumors and produced large-sized of tumors (˜90 mm³) after 31 days, whereas mice injected with MG-63 cells started to develop tumors 17 days after injection and tumor volume was small (˜10 mm³) (FIG. 5a). Then, the effect of GFRα1 deficiency on tumor formation was examined by implanting GFRα1-deficient MG-63 cells (MG-63/GFRα1 shRNA). Whereas mice injected with MG-63 cells developed tumors, injection of MG-63/GFRα1 shRNA cells into mice did not produce tumors even after 31 days (FIG. 5b).

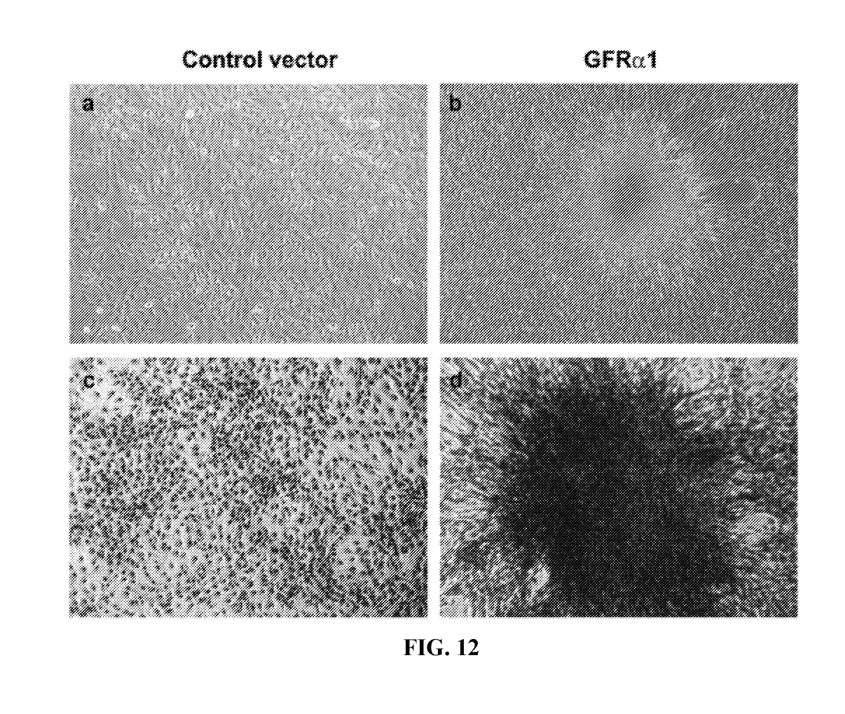

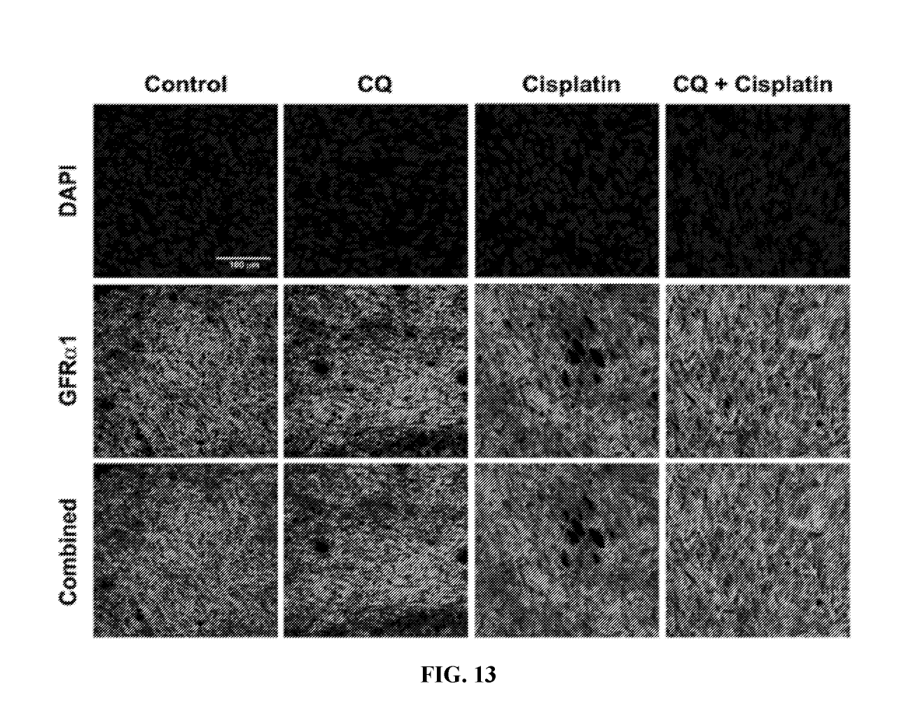

To assess whether enhanced tumor formation observed in mice injected with MG-63/GFRα1 cells resulted from GFRα1-mediated autophagy, mice injected with MG-63/GFRα1 cells were treated with PBS (control), CQ, cisplatin, or cisplatin+CQ. Treatment of mice with either CQ or cisplatin decreased tumor volume compared to PBS-treated mice, and treatment of mice with both cisplatin and CQ significantly decreased tumor volume further (FIG. 5c). The results demonstrated that only treatment with both cisplatin and CQ reduced tumor progression while single treatment with either CQ or cisplatin allowed the tumors to survive (FIG. 5c). Immunofluorescence analysis revealed that tumor cells arising from MG-63/GFRα1 cell grafted mice treated with cisplatin showed a significant increase in HMGB1 expression localized in the cytoplasm, signifying increased autophagy. Co-treatment of cisplatin with CQ significantly reduced cytoplasmic localization of HMGB1 (FIG. 5d and FIG. 13). Inhibition of autophagy with CQ during cisplatin treatment also significantly increased cisplatin-induced apoptosis in GFRα1-expressing tumors in comparison to CQ- or cisplatin-treated GFRα1-expressing tumors (FIG. 5e). Furthermore, the survival rate of MG-63/GFRα1 cell grafted mice treated with both CQ and cisplatin was significantly increased compared to mice treated only with cisplatin (FIG. 5f), indicating that inhibition of autophagy reverses GFRα1-mediated protection from cisplatin-induced apoptosis. It is noted that, among 10 MG-63/GFRα1 grafted mice treated with both CQ and cisplatin, two mice died due to cisplatin-induced liver and kidney injury, not due to tumor at day 73, indicating the potential side effects in the combined use of CQ and cisplatin as previously reported (42). Taken together, these results demonstrate that GFRα1 facilitates autophagy in response to cisplatin in vivo and this autophagic response promotes the survival of osteosarcoma tumors, suggesting that GFRα1-mediated autophagy is critical to the development of cisplatin resistance in osteosarcoma.

Contribution of GFRα1-mediated autophagy to clinicopathology of cisplatin-resistant osteosarcoma patients. The inventors further investigated GFRα1-mediated autophagy in vivo by examining tissue samples from 27 osteosarcoma patients who had received neoadjuvent and/or adjuvant therapy along with complete surgical resection. Among those cases, nine patient tissue samples displayed DAPI nuclear staining indicating survival of osteosarcoma (Table 2). From the nine samples demonstrating chemoresistance, osteosarcoma tissues before and after cisplatin treatment were processed and analyzed for GFRα1 and HMGB1 immunostaining. Four samples were positive for GFRα1 expression and only these four GFRα1-positive samples were also positive for HMGB1 expression (FIGS. 5g and 5h and Table 1). The four tissue samples were obtained from patients that received chemotherapy, including cisplatin, for 4-15 weeks. Moreover, tumors from these four osteosarcoma patients metastasized to the lungs (Table 1 and Table 2). Tissue from patients that had been treated for less than four weeks did not express GFRα1 and HMGB1 (Table 1 and Table 2). Collectively, these studies suggest a critical role for GFRα1 in cisplatin-mediated chemoresistance and metastasis.

TABLE 1
Relative expression of GFRα1 and HMGB1 and metastatic status
in osteosarcoma patients after combinational chemotherapy.

| | | | Treatment | | | |
|---|---|---|---|---|---|---|
| | | Combinational | Period | GFRα1 | HMGB1 | Metastatic |
| Case No. | Tumor Site | Treatment | (wk) | expression | Expression | status |
| 1 | Distal | IFO, DOX, | 15 | − | − | — |
| | Femur | MTX, CIS | | | | |
| 2 | Fibular | DOX, CIS | 4 | − | − | — |
| | Head | | | | | |
| 3 | Distal | MTX, VCR, | 4 | + | + | Lung |
| | Femur | CIS | | | | |
| 4 | Mandible | DOX, CIS | 5 | − | − | — |
| 5 | Distal | DOX, CIS | 10 | + | + | Lung |
| | Femur | | | | | |
| 6 | Distal | DOX, CIS | 1 | − | − | — |
| | Femur | | | | | |
| 7 | Distal | DOX, CIS | 2 | − | − | — |
| | Femur | | | | | |
| 8 | Distal | IFO, DOX, | 8 | + | + | Lung |
| | Femur | CIS | | | | |
| 9 | Proximal | DOX, CIS | 10 | + | + | Lung |
| | Femur | | | | | |

IFO: Ifosfamide,
DOX: Doxorubicin,
MTX: Methotrexate,
CIS: Cisplatin,
VCR: Vincristine

TABLE 2
Information of osteosarcoma patients.

| Case No. | Gender | Age | Diagnosis |
|---|---|---|---|
| 1 | M | 20 | Osteosarcoma, osteoblastic type |
| 2 | M | 7 | Parosteal osteosarcoma |
| 3 | M | 7 | Osteosarcoma, osteoblastic type |
| 4 | F | 76 | Osteosarcoma, osteoblastic type |
| 5 | F | 17 | Osteosarcoma, osteoblastic type |
| 6 | M | 15 | Osteosarcoma, chondroblastic type |
| 7 | M | 15 | Osteosarcoma, chondroblastic type |
| 8 | M | 20 | Osteosarcoma, osteoblastic type |
| 9 | F | 3 | Osteosarcoma, osteoblastic type |

TABLE 3
Clinicopathologic characteristics and metastatic
status of osteosarcoma patients.

| | | GFRα1 | HMGB1 |
|---|---|---|---|
| | | Expression | Expression |
| Parameter | n | n (%) | n (%) |
| Gender | | | |
| Female | 3 | 2 (66.7) | 2 (66.7) |
| Male | 6 | 2 (33.3) | 2 (33.3) |
| Age | | | |
| >20 years old | 1 | 0 (0) | 0 (0) |
| ≤20 years old | 8 | 4 (50.0) | 4 (50.0) |
| Tumor site | | | |
| Distal femur | 6 | 3 (50) | 3 (50) |
| Proximal femur | 1 | 1 (100) | 1 (100) |
| Others | 2 | 0 (0) | 0 (0) |
| Histological classification | | | |
| Osteoblastic | 6 | 4 (66.7) | 4 (66.7) |
| Chondroblastic | 2 | 0 (0) | 0 (0) |
| Others | 1 | 0 (0) | 0 (0) |
| Treatment period (weeks) | | | |
| <4 | 2 | 0 (0) | 0 (0) |
| 4-15 | 7 | 4 (57.1) | 4 (57.1) |
| Metastatic status | | | |
| Non-metastatic | 5 | 0 (0) | 0 (0) |
| Metastatic (lung) | 4 | 4 (100) | 4 (100) |

B. Methods

Human tissue. Chonnam National University Hwasun Hospital (Gwangju, Chonnam, South Korea), a member of the National Biobank of Korea supported by the Ministry of Health, Welfare, and Family Affairs, provided the biospecimens for this study. All specimens were obtained with informed consent under Chonnam National University School of Medicine Institutional Ethics Review Board-approved protocol.

Cell culture. Human osteosarcoma cell lines MG-63 and U-2 OS, human embryonic kidney cell line 293T, human fibrosarcoma cell line HT-1080, human pancreatic cancer cell line MIA PaCa-2, and mouse embryonic fibroblast cell line NIH/3T3 were purchased from the American Type Culture Collection (Rockville, Md.). Cell lines were maintained in Dulbecco's Modified Eagle's Medium (MG-63, 293T, MIA PaCa-2 and NIH/3T3), Eagle's Minimum Essential Medium (HT-1080) or McCoy's 5a Modified Medium (U-2 OS) supplemented with 10% heat-inactivated fetal bovine serum (FBS) and 1% penicillin/streptomycin. Cells were maintained in 5% CO₂-humidified atmosphere at 37° C.

Generation of stable cell lines. A human pLenti/GFRα1 expression vector and empty control vector were purchased from GeneCopoeia (Rockville, Md.). Viral packaging using 293T cells and titration of the full lentiviral vector were performed by using the Invitrogen Gateway System and ViraPower Lentiviral Expression System. The presence of GFRα1 was confirmed by PCR, and correct insertion of the clone was further confirmed by sequencing. Lentiviral transduction, expression, and titration were performed using HT-1080 cells. The packed virus was concentrated by ultracentrifugation (20,000×g for 2 hr at 4° C.) using Centricon filters (Millipore, Billerica, Mass.). For establishing stable MG-63, U-2 OS or NIH/3T3 cell lines overexpressing GFRα1, cells were infected with lentivirus containing the pLenti/control or the pLenti/GFRα1 vector. Cells were then incubated in medium containing 400 μg/ml of G418 for 4-5 weeks. GFRα1-specific shRNA and control shRNA were purchased from Santa Cruz Biotechnology (Santa Cruz, Calif.). MG-63 and U-2 OS cells were transfected with either GFRα1-specific shRNA or control shRNA using Lipofectamine® 2000 (Life Technologies, Grand Island, N.Y.) and cultured in selection medium containing 100 μg/ml of puromycin for 4-5 weeks to generate stable GFRα1 knockdown cell lines.

Generation of cisplatin-resistant cell lines. Cisplatin-resistant (CIS^{R}) sub-lines were derived from MG-63 parental cell line by continuous exposure to cisplatin following initial dose-response studies of cisplatin (1 μM-1 mM) over 24 hr from which IC50 values were obtained. Initially, CIS^{R} sub-line was treated with cisplatin (1 μM) for 72 hr. The media was removed and cells were allowed to recover for 72 hr. Subsequently, the resistant sub-lines were generated by gradual exposure to low to high-dosage of cisplatin (1-50 μM). This development period was carried out for approximately 6 months, and then cells were maintained continuously in the presence of cisplatin at these new IC50 concentrations for 2 months.

Reagents and Antibodies. Dimethyl sulfoxide (DMSO) and N,N-Dimethylformamide, Cis-Diamineplatinum(II) dichloride (cisplatin), bafilomycin A1 (Baf), 3-methyladenine (3-MA) and choloroquine diphosphate salt (CQ) were purchased from Sigma-Aldrich (St Louis, Mo.). Methotrexate and doxorubicin were purchased from Santa Cruz Biotechnology. The following antibodies were used: anti-GFRα1, anti-phospho-mTOR, anti-mTOR, anti-c-Src, anti-caspase-3, and anti-β-actin from Santa Cruz Biotechnology; anti-HMGB1 from Abcam (Cambridge, Mass.); anti-phospho-Src, anti-beclin 1, anti-phospho-AMPK, anti-AMPK, anti-LC3B, and anti-phosphop70 S6 Kinase from Cell Signaling Technology (Beverly, Mass.).

Quantitative real-time (RT)-PCR (qPCR). Total RNA was collected and isolated from cell lines using the RNeasy system according to the manufacturer's protocol (Qiagen, Hilden, Germany). cDNA was generated from a 30 μl reaction containing total RNA (3 μg), reverse transcriptase and oligo dT primers (Promega, Madison, Wis.). Real-time amplification of GFRα1 and NFκB p50 cDNA was performed with a LightCycler® 96 Instrument (Roche, Indianapolis, Ind.) using SYBR® Mastermix FastStart Essential DNA Green Master (Roche). Gene-specific human primer pairs of 18S rRNA, GFRα1 and NFκB p50 were designed using The Primer Express® software v3.0.1 (Applied Biosystems, Grand Island, N.Y.). The following primers were synthesized and used for qPCR: q18S rRNA sense primer 5′-GAGGATGAGGTGGAACGTGT-3′ (SEQ ID NO:1) and antisense primer 5′-TCTTCAGTCGCTCCAGGTCT-3′ (SEQ ID NO:2) (designed to amplify a 166-bp region); qGFRα1 sense primer 5′-TCCAATGTGTCGGGCAATAC-3′ (SEQ ID NO:3) and antisense primer 5′-GGAGGAGCAGCCATTGATTT-3′ (SEQ ID NO:4) (designed to amplify a 106-bp region); qNFκB p50 sense primer 5′-AAGCACAAAAAGGCAGCACT-3′ (SEQ ID NO:5), antisense primer 5′-TGCCAATGAGATGTTGTCGT-3′ (SEQ ID NO:6) (designed to amplify a 197-bp region). The amplification conditions consisted of one cycle at 95° C. for 10 sec followed by 48 cycles at 95° C. for 5 sec and a 60° C. annealing step for 20 sec. Amplification was followed by a melting curve analysis to verify the correct size of the amplicon. A negative control without cDNA was run with every PCR to assess the specificity of the reaction. An analysis of the data was performed using LightCycler® 96 software 1.1 (Roche).

Semi-quantitative RT-PCR. Gene-specific human primer pairs of GFRα1, GDNF and GAPDH were designed using The Primer Express® software v3.0.1 (Applied Biosystems). The following primers were synthesized and used for PCR: GFRα1 sense primer 5′-TGTCAGCAGCTGTCTAAAGG-3′ (SEQ ID NO:7) and antisense primer 5′-CTTCTGTGCCTGTAAATTTGCA-3′ (SEQ ID NO:8) (designed to amplify a 387-bp region); GDNF sense primer 5′-CCAACCCAGAGAATTCCAGA-3′ (SEQ ID NO:9) and antisense primer 5′-AGCCGCTGCAGTACCTAAAA-3′ (SEQ ID NO:10) (designed to amplify a 150-bp region); GAPDH sense primer 5′-TGACCACAGTCCATGCCATC-3′ (SEQ ID NO:11) and antisense primer 5′-TTACTCCTTGGAGGCCATGT-3′ (SEQ ID NO:12) (designed to amplify a 494-bp region). PCR reactions were optimized to 94° C. for 3 min, 28 amplification cycles at 94° C. for 30 sec, 58° C. for 30 sec, 72° C. for 30 sec, and a final extension of 10 min at 72° C. Amplified products were resolved on 1.5% agarose gels and visualized by ethidium bromide staining.

Small interfering RNA (siRNA)-based Experiments. Human GFRα1 siRNA, Beclin 1 siRNA, HMGB1 siRNA, AMPK siRNA, c-Src siRNA, NFκB p50 siRNA, APE siRNA and Negative (control) siRNA were purchased from Santa Cruz Biotechnology. Cell lines were transiently transfected with siRNA duplexes using Lipofectamine® RNAiMAX (Life Technologies) according to manufacturer's instructions.

Cell viability assays. WST-1 cell viability assay reagent was purchased from Roche. Equal numbers of cells were seeded in triplicate wells in 48-well plates and maintained in growth medium containing 10% FBS). Cells were cultured with PBS, GDNF (50 ng/mL), GDNF (50 ng/ml)+cisplatin (20 μM), or cisplatin (20 μM) in the presence of serum for 24 hr. Cells were also preincubated with inhibitors (3-MA, PP1, Compound C, Baf and CQ) for 1 hr before stimulating with PBS, GDNF, GDNF+cisplatin, or cisplatin. WST-1 reagent was then treated to the cells at the indicated times and incubated for 2 hr at 37° C. Cell viability was determined by measuring the absorbance after adding WST-1. The spectrophotometric absorbance of the samples was measured using an Ultra Multifunctional Microplate Reader (Tecan, Durham, N.C.) at 450 nm. Experiments were done at least in triplicate using separate cultures.

Apoptosis Assays. Floating and trypsin-detached cells were collected and washed once with ice-cold PBS, followed by FITC Annexin V Apoptosis Detection Kit (BD Biosciences, San Diego, Calif.). Apoptotic cells were analyzed using a FACSCalibur flow cytometer with CellQuest software (Becton Dickinson, Franklin Lakes, N.J.). The results represent the means of triplicate determinations in which a minimum of 10,000 cells was assayed for each determination. Caspase 3 activity was analyzed by the Colorimetric CaspACE™ Assay System (Promega, Madison, Wis.) according the manufacturer's instructions. The degree of apoptosis in tissue was assessed with terminal deoxynucleotidyl transferase deoxyuridine triphosphate nick end labeling (TUNEL) assay from ApopTag Plus Peroxidase In Situ (Millipore, Billerica, Mass.).

FACS analysis. Cells were harvested by trypsinization, washed with PBS, fixed in 70% ethanol, washed with PBS and then incubated with 0.02 mg/ml of propidium iodide including RNase A. Cells were analyzed using a FACSCalibur flow cytometer with CellQuest software (Becton Dickinson, Franklin Lakes, N.J.).

TUNEL assays. The apoptotic index in tissue was determined by the TUNEL assay. The sections were stained with ApopTag Plus Peroxidase In Situ. The sections were incubated at 60° C. overnight. Sections were deparaffinized in xylene for an hour and rehydrated with reduced alcohol series (100, 95, 90, 70, and 50%). The slides were incubated with 20 μg/ml of proteinase K (Invitrogen, Camarillo, Calif.) for 15 min. Washing with PBS was performed in every stage. Endogenous peroxidase activity was blocked with 3% H2O₂. After washing with PBS, the sections were incubated with equilibration buffer for 10-15 min, and with terminal deoxynucleotidyl transferase (TdT) enzyme (77 μl reaction buffer +33 μl TdT enzyme mix [1 μl TdT enzyme]) for 1 hr at 37° C. Stop/wash buffer (1:10) was applied for 10 min at room temperature, and the slides were incubated with antidigoxigenin conjugate for 30 min. After washing with PBS 3 times for 5 min, the sections were stained with DAB components to detect TUNEL-positive cells, and then they were counterstained with methyl green. Data were quantified and analyzed with Image-J software (National Institutes of Health, Bethesda, Md.).

Autophagy assays. Formation of autophagic vesicles was monitored by endogenous or exogenous LC3-II aggregation in cell lines by LC3B antibody or mRFP-GFP-LC3 plasmid provided by Dr. Myung Shik Lee from Samsung Medical Center, Sungkyunkwan University School of Medicine (Seoul, Korea). The protein levels of LC3-II were determined by western blotting after transfection with mRFP-GFP-LC3 into cell lines. Staining of Acidic vesicular organelles (AVOs) by acridine orange was performed according to published procedures (43). Acridine orange (Polysciences, Warrington, Pa.) was added at a final concentration of 0.5 mg/ml for 15 min. All fluorescence images including AVOs and LC3 puncta are confocal images acquired with a LSM510 laser-scanning microscope (Carl Zeiss, Gottingen, Germany). Data were quantified and analyzed with Image-J software. LC3-labeled puncta were defined as bright dots >1.5 SD above the mean cytosolic fluorescence. At least three individual experiments were performed, and at least 40 (for AVOs) or 20 (for LC3 puncta) sections were analyzed (15).

Transmission Electron Microscopy. Cells were fixed with 2% paraformaldehyde and 2% glutaraldehyde in 0.1 M phosphate buffer (pH 7.4) and then postfixed with 1% OsO₄ for 2 hr. The cells were dehydrated with increasing concentrations of alcohol (30, 50, 70, 90, and 100%), infiltrated with LR White resin two times for 1 hr, and embedded in LR White resin. The solidified blocks were cut into 60-nm thicknesses and stained with uranyl acetate and lead citrate. Samples were observed under a transmission electron microscope (Hitachi H-7600; Hitachi, Tokyo, Japan). Ten fields of images were selected and the autophagic vacuoles were quantified as previously described.

Colony-forming assays and Crystal Violet Staining. MG-63 and MG-63 Resistant (MG-63-CIS^{R}) cells, pLenti/GFRα1-, pLenti/empty vector-, shGFRα1-, and control shRNA-transfected MG-63 cells were cultured in DMEM supplemented with 10% FBS. For colony-formation assays, an equal numbers of cells from each individual clone were plated onto 12-well plates. After 24 hr, cells were incubated with cisplatin, or cisplatin+autophagy inhibitors, respectively. All medium was exchanged every 3 days under the same condition. Colonies were visualized after 10 days. The cells were washed with PBS, fixed in 4% PFA for 5 min, and again washed with PBS. Fixed cells were stained with 0.05% crystal violet in distilled water for 1 hr, washed with distilled water and then drained. Images of stained colonies were scanned using an Epson scanner (GT9700F, Tokyo, Japan) and then counted with Image-Pro Plus 5.1 software (Media Cybernetics, Bethesda, Md.). The experiments were repeated in triplicate.

Focus forming assays. NIH/3T3 cells (1×10⁶) were plated onto 60-mm dishes and incubated overnight to form a monolayer. The next day, NIH/3T3 cells (1×10³) stably transfected with an expression vector encoding GFRα1 were plated on control NIH/3T3 cells. Medium was exchanged every 2 days and the foci was quantitated after 7-10 days. Cells stably transfected with the empty expression vector provide the negative control for this assay. All of cells were stained with crystal violet (0.5% in 20% ethanol) for foci.

Immunoblotting. Cells were washed with PBS and lysed in RIPA Buffer (Pierce, Rockford, Ill.). The protein content was determined using a dye-binding microassay (Bio-Rad, Hercules, Calif.), and 10-50 μg protein per lane was electrophoresed on 4-12% SDS polyacrylamide gels. Proteins were blotted onto Hybond ECL membranes (Amersham Pharmacia Biotech, Piscataway, N.J.). Two protein ladders were used for molecular weight determination (GenDEPOT, Barker, Tex.). The blotted proteins were detected using an enhanced chemiluminescence detection system (iNtRON, Seoul, Korea). Data were quantified and analyzed with Image-J software.

Immunocytochemistry. Osteosarcoma cells (2×10⁴) were seeded on 60 μ-Dish 35 mm high (ibidi, GmbH, Am Klopferspitz, Germany). Next day, cells were fixed in 4% PFA for 20 min. After being washed by PBS, and then incubated in 0.04% of Triton X-100. After being washed by PBS, they were incubated in 0.03% of BSA for 10 min at room temperature. Then, antibodies were applied overnight at 4° C. and washed for 1 hr in PBS. Alexa Fluor 488- or 647-conjugated secondary antibodies were applied 1 hr at 4° C. and washed for 1 hr in PBS. Slides were rinsed in PBS, mounted in VECTASHIELD™ (Vector Laboratories, Burlingame, Calif.) and sealed with clear nail varnish. Images were taken by confocal microscopy). LC3 puncta or APE was quantified and analyzed with Image-J. In the above analysis, there was no discrepancy between the two observers regarding the patterns of biomarker expression and the scores assigned to analyzed sections.

Immunohistochemistry. Triplicate core biopsies of 1 mm were taken from each donor paraffin block and arrayed. The sections (5 μm thick) were deparaffinized and underwent hematoxylin and eosin stain, and immunohistochemistry. After antigen retrieval by 10 mM sodium citrate (pH 6.0), sections were incubated with rabbit anti-HMGB1 and mouse anti-GFRα1 antibodies for 24 hr at 4° C. Sections were followed by incubation with biotinylated secondary antibodies, and then antibody labeling was visualized by using the VECTASTAIN™ ABC Systems (Vector Laboratories). For immunofluorescence, immunohistochemistry of patient tissues and mouse tumors are incubated with the Alexa Fluor 488- and 647-conjugated secondary antibodies (Invitrogen), and nuclei were counterstained with DAPI (Sigma-Aldrich) Immunofluorescence was detected by confocal microscopy. In patient tissue studies, immunoreactivity was not determined by scoring according to the staining intensity (0, non; 1, weak; 2, moderate; 3, strong) of immunolabeling and percent positive cells (0, <5%; 1, 6˜25%; 2, 26˜50%; 3, 50˜75%; 4, >76%) because all of metastatic patient tissues between 4 weeks and 10 weeks after chemotherapy including cisplatin showed the robust immunoreactivity of GFRα1 or HMGB1, while other patient tissues (below 4 weeks or over 10 week after chemotherapy w/wo cisplatin) never show the immunoreactivity of them. Thus, marked plus (+) was considered to be positive immunoreactivity, while that minus (−) was considered to be negative immunoreactivity (not detected). In the above analysis, there was no discrepancy between the two observers regarding the patterns of biomarker expression and the scores assigned to analyzed sections. Data were quantified and analyzed with Image-J software.

Tumor formation in nude mice. The mice used in this study were 6-week-old female BALB/c nude mice purchased from Orient Bio Inc. (Seongnam, Korea). They were housed in our pathogen-free facility and handled in accordance with standard-use protocols and animal welfare regulations. All study protocols were approved by the institutional Animal Care and Use Committee at Chonnam National University. MG-63 cells stably transfected with the indicated Control shRNA, GFRα1 shRNA, pLenti/GFRα1 expression viral vector, or pLenti/control viral vector were harvested and resuspended in PBS. Then MG-63 cells (1×10⁶) were injected subcutaneously into the right flank of a BALB/c nude mouse. The tumor size was measured with a caliper every 3 or 4 days. After 31-days subcutaneous injection, mice with GFRα1 expressing tumor cells were administrated with PBS, CQ (60 mg/kg), cisplatin (3 mg/kg), and cisplatin+CQ into the peritoneum once a week. The tumor size was measured with a caliper every 4 days. Tumor weights were calculated from caliper measurements of tumor dimensions in mm using the formula for a prolate ellipsoid: (L×W²)/2 where L is the longer of the two-measurements.

Statistical analysis. Survival data was analyzed by the Kaplan-Meier and log-rank test with GraphPad Prism v5.04 (GraphPad Software, La Jolla Calif.). Plating efficiency (PE, %)=(Number of colonies counted/Number of cells plated)×100. Survival fraction (SF, %)=(PE of cisplatin-treated clone/PE of non-treated control clone). All values are expressed as the mean±standard deviation (SD). Where indicated, statistical analyses were performed using two-tailed Student's t-test. **p<0.05 was considered statistically significant.

### REFERENCES

1. Ottaviani and Jaffe, Cancer Treat Res 152, 3-13 (2009).
2. Collins et al. J Clin Oncol 31, 2303-2312 (2013).
3. Kansara et al. Nat Rev Cancer 14, 722-735 (2014).
4. Meyers, et al. J Clin Oncol 23, 2004-2011 (2005).
5. Basu and Krishnamurthy, J Nucleic Acids 2010 (2010).
6. He et al. Oncol Lett 7, 1352-1362 (2014).
7. Lum et al. Nat Rev Mol Cell Biol 6, 439-448 (2005).
8. Boya et al. Nat Cell Biol 15, 713-720 (2013).
9. Levine and Klionsky, Dev Cell 6, 463-477 (2004).
10. Mizushima, Genes Dev 21, 2861-2873 (2007).
11. Kondo et al., Nat Rev Cancer 5, 726-734 (2005).
12. Levine, Autophagy 2, 65-66 (2006).
13. Mathew et al., Nat Rev Cancer 7, 961-967 (2007).
14. Chen and Karantza-Wadsworth, Biochim Biophys Acta 1793, 1516-1523 (2009).
15. Liu et al., Leukemia 25, 23-31 (2011).
16. Sui et al., Cell Death Dis 4, e838 (2013).
17. Ding et al., Clin Cancer Res 17, 6229-6238 (2011).
18. Liu et al., Autophagy 7, 112-114 (2011).
19. Kang et al., Cell Death Differ 17, 666-676 (2010).34
20. Airaksinen and Saarma, Nat Rev Neurosci 3, 383-394 (2002).
21. Treanor et al., Nature 382, 80-83 (1996).
22. Bespalov and Saarma, Trends Pharmacol Sci 28, 68-74 (2007).
23. Paratcha and Ledda, Trends Neurosci 31, 384-391 (2008).
24. Esseghir et al., Cancer Res 67, 11732-11741 (2007).
25. Wu et al., BMC Cancer 13, 34 (2013).
26. Cavel et al., Cancer Res 72, 5733-5743 (2012).
27. Kim et al., Mol Cell Biol 29, 2264-2277 (2009).
28. Mizushima et al., Cell 140, 313-326 (2010).
29. Kimura et al., Autophagy 3, 452-460 (2007).
30. Poteryaev et al., Febs Letters 463, 63-66 (1999).
31. Meley et al., J Biol Chem 281, 34870-34879 (2006).
32. Ahn and Lee, Cancer Lett 310, 188-197 (2011).
33. Zou et al., J Biol Chem 278, 34003-34010 (2003).
34. Mizrachy-Schwartz et al., J Biol Chem 286, 15268-15277 (2011).
35. Harhaji-Trajkovic et al., J Cell Mol Med 13, 3644-3654 (2009).
36. Degenhardt et al. Cancer Cell 10, 51-64 (2006).
37. Ng et al., J Clin Neurosci 16, 427-436 (2009).
38. Hansford and Marshall, Neurosci Lett 389, 77-82 (2005).
39. Mulligan, Nat Rev Cancer 14, 173-186 (2014).
40. Ferrari et al., J Clin Oncol 30, 2112-2118 (2012).
41. Huang et al., Cancer Res 72, 230-238 (2012).
42. Yang et al., Technol Cancer Res Treat 9, 161-169 (2010).
43. Paglin et al., Cancer Res 61, 439-444 (2001).

1. A method of treating a subject with cancer comprising:
(a) identifying a subject susceptible or resistant to a platinum chemotherapy by contacting a sample from the subject comprising a cancer cell with a detection reagent specific for GFRα1 molecule forming a complex between the detection reagent and a GFRα1 molecule;
(b) determining the level of the GFRα1 molecule in the sample; and
(c) administering (i) a platinum chemotherapy if the GFRα1 molecule levels indicate the cancer is sensitive to platinum chemotherapy or (ii) a non-platinum chemotherapy if the GFRα1 molecule levels indicate resistance to cisplatin chemotherapy.

2. The method of claim 1, further comprising administering an autophagy inhibitor.

3. The method claim 1, wherein the cancer is bone, pancreatic, kidney, stomach, brain, colon, skin, lung, bladder, prostate, uterine, cervical, breast or ovarian cancer.

4. The method of claim 1, wherein the cancer is osteosarcoma.

5. The method of claim 1, wherein the GFRα1 molecule is a GFRα1 nucleic acid.

6. The method of claim 1, wherein the GFRα1 molecule is a GFRα1 polypeptide.

7. The method of claim 1, wherein the platinum chemotherapy is selected from carboplatin, cisplatin, oxaliplatin, BBR3464, or satraplatin.

8. Use of autophagy inhibitors to ameliorate chemoresistance in GFRα1 expressing cancer comprising administering an effective amount of the autophagy inhibitor to a subject identified as having GFRα1 molecule levels indicative of resistance to cisplatin chemotherapy.

9. The use according to claim 8, wherein the autophagy inhibitor is selected from Chloroquine, Bafilomycin A1, 3-Methyladenine, Hydroxychloroquinine, LY 294002, Bay K 8644, Concanamycin A, DBeQ, E 64d, GW 4064, ML 240, Nocodazole, Pepstatin A, Spautin 1, Vinblastine, Wortmanin, or Xanthohumol.

10. (canceled)

11. A method of determining if a subject has become or is at risk of becoming chemoresistant, comprising:
(a) obtaining a biological sample from the subject; and
(b) measuring the level of GFRα1, wherein an increased level of GFRα1 is indicates that the subject is or will become chemoresistant.

12. The method of claim 11, wherein the subject is chemoresistant to a platinum based chemotherapeutic.

13. The method of claim 12, wherein the platinum based therapeutic is selected from the group consisting of: Carboplatin, Cisplatin, Oxaliplatin, BBR3464, and Satraplatin.

14. The method of claim 11, wherein the subject has a cell proliferative disorder.

15. The method of claim 14, wherein the cell proliferative disorder is cancer.

16. The method of claim 15, wherein the cancer is bone cancer.

17. The method of claim 16, wherein the bone cancer is osteosarcoma.


SEQUENCE LISTING (12 sequences)

SEQ ID NO: 1 - DNA, 20 bp, Artificial Sequence
  Synthetic Primer
gaggatgaggtggaacgtgt

SEQ ID NO: 2 - DNA, 20 bp, Artificial Sequence
  Synthetic Primer
tcttcagtcgctccaggtct

SEQ ID NO: 3 - DNA, 20 bp, Artificial Sequence
  Synthetic Primer
tccaatgtgtcgggcaatac

SEQ ID NO: 4 - DNA, 20 bp, Artificial Sequence
  Synthetic Primer
ggaggagcagccattgattt

SEQ ID NO: 5 - DNA, 20 bp, Artificial Sequence
  Synthetic Primer
aagcacaaaaaggcagcact

SEQ ID NO: 6 - DNA, 20 bp, Artificial Sequence
  Synthetic Primer
tgccaatgagatgttgtcgt

SEQ ID NO: 7 - DNA, 20 bp, Artificial Sequence
  Synthetic Primer
tgtcagcagctgtctaaagg

SEQ ID NO: 8 - DNA, 22 bp, Artificial Sequence
  Synthetic Primer
cttctgtgcctgtaaatttgca

SEQ ID NO: 9 - DNA, 20 bp, Artificial Sequence
  Synthetic Primer
ccaacccagagaattccaga

SEQ ID NO: 10 - DNA, 20 bp, Artificial Sequence
  Synthetic Primer
agccgctgcagtacctaaaa

SEQ ID NO: 11 - DNA, 20 bp, Artificial Sequence
  Synthetic Primer
tgaccacagtccatgccatc

SEQ ID NO: 12 - DNA, 20 bp, Artificial Sequence
  Synthetic Primer
ttactccttggaggccatgt